(19) United States
(12) Patent Application Publication
(10) Pub. No.: US 2023/0365632 A1
(43) Pub. Date: Nov. 16, 2023

(54) A GROUP OF PEPTIDE DERIVATIVE OMICSYNINS WITH ANTIVIRAL ACTIVITY AND USES THEREOF

(71) Applicant: INSTITUTE OF MEDICINAL BIOTECHNOLOGY CHINESE ACADEMY OF MEDICAL SCIENCES, Beijing (CN)

(72) Inventors: Shuyi SI, Beijing (CN); Bin HONG, Beijing (CN); Yuhuan LI, Beijing (CN); Hongmin SUN, Beijing (CN); Jiandong JIANG, Beijing (CN); Xingxing LI, Beijing (CN); Minghua CHEN, Beijing (CN); Rongmei GAO, Beijing (CN); Yongsheng CHE, Beijing (CN); Xin ZHEN, Beijing (CN); Liyan YU, Beijing (CN); Hongyu LIU, Beijing (CN); Ming ZHONG, Beijing (CN); YeXiang WU, Beijing (CN); Yuanyuan SHI, Beijing (CN)

(21) Appl. No.: 18/030,107

(22) PCT Filed: Feb. 10, 2021
(86) PCT No.: PCT/CN2021/076524

§ 371 Date: Apr. 4, 2023

(30) Foreign Application Priority Data
Feb. 17, 2020 (CN) 202010097607.1

(51) Int. Cl. C07K 14/36; A61P 31/12

(52) U.S. Cl. CPC C07K 14/36; A61P 31/12

(57) ABSTRACT

This disclosure related to a group of peptide derivative Omicsynins with antiviral activity against influenza virus and coronavirus. It also related uses of these derivatives, the chemical formula of the peptide derivative Omicsynins is shown in formula (1):

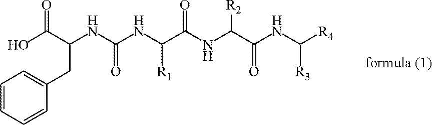

R1—R4 are shown in the following table.

| | | | |
|---|---|---|---|
| NO. | substituent1 | substituent2 | substituent3 |
| R₁ | 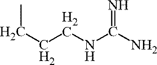 | 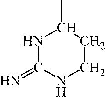 | 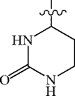 Basic amino-acid side chains including lysine, histidine, citrulline residues, and etc. |
| R₂ | 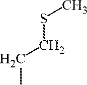 | —CH(CH₃)₂, —CH(CH₃)CH₂CH₃ 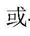 —CH₂CH(CH₃)₂ | Neutral amino-acid side chain including tryptophan, serine, threonine, cysteine residues and etc. |
| R₃ | 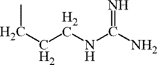 | 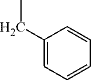 | 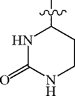 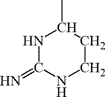 R₃ including tyrosine, lysine, histidine, citrulline residues, and etc. |
| R₄ | —CH₂OH | —CHO | R₄ including —CH₂NH₂, —CH═NH,— CH═NOH, —COOH, —COOR₅ (R₅ represents alkyl groups containing 1-3 carbons), -CONH₂ and etc. |

### TECHNICAL FIELD

The disclosure relates to the field of biomedicine, in particular to a group of peptide derivative Omicsynins with antiviral activity and uses thereof.

### BACKGROUND

Severe viral infections are diseases that seriously endanger human beings. Among them, the acute respiratory syndrome coronavirus (SARS) is noticed for its strong infectiousness and high lethality in the new century. From the autumn of 2002 to the spring of 2003, the atypical pneumonia caused by the SARS coronavirus that broke out in Guangdong province of China infected 8,458 people in 37 countries, with 807 deaths and a mortality rate of about 10%. In 2012, acute respiratory diseases caused by Middle East respiratory syndrome coronavirus (MERS coronavirus, MERS-CoV) were prevalent in Saudi Arabia and parts of the Middle East, which led to pneumonia, renal failure and death. Official statistics showed that it infected 2,943 people resulting in 858 deaths (including 38 in South Korea). As of Feb. 9, 2021, COVID-19 has infected more than 100 million people and killed more than 2 million people, and there is an urgent need for safe and effective broad-spectrum antiviral drugs, especially the effective treatment of novel coronavirus with anti-coronavirus drugs because of the serious epidemic.

Recently, the results of the whole gene sequencing of the 2019-nCoV coronavirus showed that the virus was only 79% homologous to the SARS coronavirus and belonged to a new coronavirus different from the SARS coronavirus which have been published by Chinese CDC (Centers for Disease Control and Prevention) in “New England Medicine” on Jan. 24, 2019 and “The Lancet” on January 29 respectively. The 2019-nCoV coronavirus genome encodes regions for proteins including 29844 bases, encoding 12 proteins: 1ab, S, 3, E, M, 7, 8, 9, 10b, N, 13, and 14. Among them, 1ab is a gene encoding a non-structural polyprotein composed of 7096 amino acids, S is a gene encoding a spike protein, and also contains other envelope proteins such as E, M and N proteins. Generally speaking, the non-structural protein of 7096 amino acids encoded by 1ab is cleaved by virus-encoded proteases 3CLpro and PLpro to form 16 nonstructural proteins (NSPs), most of which are involved in the formation of viral replication complexes. According to the research on drug targets of SARS coronavirus and Middle East respiratory syndrome coronavirus (MERS) and the homology comparison study of 2019-nCoV coronavirus genomes, the key drug targets that can be exploited by 2019-nCoV include the interaction of the spike protein with angiotensin-converting enzyme 2 (ACE2) on the human cell membrane, that is, the entry mechanism, the RNA-dependent RNA polymerase RdRp, and Cysteine proteases 3CLpro and PLpro responsible for hydrolyzing a polyprotein consisting of 7096 amino acids into a functional protein.

When coronavirus, influenza virus, parainfluenza virus and other respiratory related viruses enter respiratory epithelial cells, they all need to use the protease of the host cell to cleave and activate viral proteins before they can enter the cells for replication, therefore, inhibitors of these host cell-encoded proteases may have broad-spectrum antiviral activity against these respiratory-associated viruses. More importantly, antiviral drugs targeting host cell proteases can also effectively avoid virus escape mutation.

Microbial-derived natural products are the main source of new anti-infective antibiotics.

According to statistics, about 10% of the microbial metabolites have anti-virus activities among the existing 25,000 microbial secondary metabolites with biological activities, such as anti-syncytial virus drug Ribavirin, the broad-spectrum antiviral antibiotics Spongouridine, Uracil 1-β-D-arabinofuranoside and vidarabine, as well as Fosfomycin and Formycin, which are derived from microbial natural products or the transformation of microbial natural products.

It has always been a research hotspot to find new drugs lead compounds from natural products, compared with secondary metabolites of animals and plants, microbial secondary metabolites have greater value for developing and utilization because they are more sustainable and friendly for the ecological environment.

As early as the 1960s, researchers at the Institute of Medical Biotechnology isolated and screened a strain of Streptomyces sp. CPCC 200451 with good antiviral activity from soil samples in southern China. The effective components were obtained from the fermentation broth of CPCC 200451 strain by using some classical screening methods and ion exchange resin column chromatography. It also has been used in clinical trials as an anti-human influenza virus drug, and it has an excellent effect to reduce high fever by intranasal instillation of the extract solution preparation. In the study, it was also found that the effective component showed high sensitivity to a variety of viruses, such as influenza virus, coronavirus, Newcastle disease virus, etc., however due to the limits of experimental conditions and separation methods at that time, as well as the existences of strong acid and other severe purification conditions, the stable and exact pharmacological components of Streptomyces sp. CPCC 200451 cannot be obtained, and the structure of the antiviral active components cannot be determined.

With the rapid development of microbial whole-genome DNA sequencing technology, more and more microbial genomes have been sequenced and information sharing has been achieved. In addition, a series of advanced technologies such as bioinformatics and molecular biology are widely used in the field of genome research, which has greatly promoted the process of mining microbial genetic resources. Studies have shown that biosynthetic genes of microbial secondary metabolites are often clustered and highly conserved. Secondary metabolic gene clusters can be found and analyzed, the structure and physicochemical properties of metabolites can be inferred, and the isolation and purification of target compounds can also be guided through bioinformatics analysis of microbial genomes. The rise of bioinformatics not only provides new opportunities for the development of microbial drugs, but also plays a very important role in the discovery of new microbial secondary metabolites. And it also provides us with a new scientific way to solve the problem of identification of antiviral active compounds produced by CPCC 200451.

With the advent of the “post-genome era”, high-throughput sequencing-based transcriptomics, metabolomics and other omics technologies have emerged one after another and have been widely used. Transcriptomics is the study of entire set of transcripts produced by the organisms in a certain functional state.

And now, the transcriptome is widely understood to mean the complete set of all the messenger ribonucleic acid (mRNA) molecules expressed in prokaryotic organism. The changes of the biosynthetic gene expression can be obtained by comparing the transcriptomes of microorganisms under different fermentation conditions. Combined with analysis techniques such as genome sequence and bioinformatics, it is helpful to locate the biosynthetic gene clusters of target metabolites. Metabonomics refers to the dynamic conditions of endogenous metabolites in organisms. Since microorganisms can produce different secondary metabolites under different fermentation conditions, the diversity of metabolites is attributed to the diversity of biosynthetic genes. The combination of genomics, transcriptomics and metabolomics analysis can not only detect the differential metabolites from the appearance, but also explain the reasons for the changes of metabolites at the gene level. Therefore, it can help us find activity-related biosynthetic gene clusters and metabolites, and guide the separation, purification and structural elucidation of target compounds to analyze the changes in the transcriptome and metabolome of microorganisms under active and inactive fermentation conditions, that is, activity-oriented comparative transcriptome and metabolome analysis.

In order to find out the effective components with antiviral activity in Streptomyces sp. CPCC 200451, we took the whole genome information of Streptomyces sp. CPCC 200451 as the starting point of the research, searched for related biosynthetic gene clusters through genomic bioinformatics analysis, identified the biosynthetic gene clusters of CPCC 200451 activity-related compounds by activity-oriented comparative transcriptomic data analysis, determined the biosynthetic gene clusters which were responsible for the active components generation through molecular biology technologies and genetic operations such as knockout and overexpression of key genes in the target gene cluster were performed. In addition, the structural characteristics related to active compounds were obtained to help the separation and structure determination of target products through activity-oriented comparative metabolomic analysis, the components with antiviral activity from the fermentation of Streptomyces sp. CPCC 200451 were finally clarified and the active compounds were separated and obtained by comprehensively using bioinformatics, chemical separation and other technical methods. At present, some of the antiviral active compounds produced by Streptomyces sp. CPCC 200451 are known protease inhibitors, such as antipain and chymostatin. Maybe this is the reason for its antiviral activity against a variety of respiratory-associated viruses, especially coronaviruses and influenza viruses.

### SUMMARY

The present disclosure provides a group of peptide derivative Omicsynins with antiviral activity.

And also provides use of said peptide derivative Omicsynins in the manufacture of anti-virus medicament, the chemical formula of the peptide derivatives is shown in formula (1):

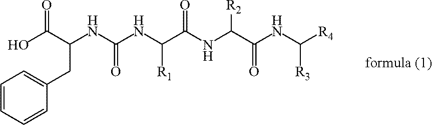

R₁-R₄ are shown in the following table:

| | | | |
|---|---|---|---|
| NO. | substituent 1 | substituent 2 | substituent 3 |
| R₁ | 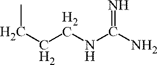 | 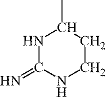 | Basic amino-acid side chains including lysine, histidine, citrulline |
| | | | 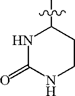 residues, and etc. |
| R₂ | 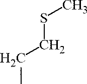 | —CH(CH₃)₂, — CH(CH₃)CH₂CH₃ [symbol] —CH₂CH(CH₃)₂ | Neutral amino-acid side chain including tryptophan, serine, threonine, cysteine residues and etc. |
| | 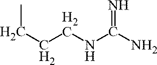 | 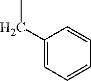 | R₃ including tyrosine, lysine, histidine, citrulline residues, |
| R₃ | | | 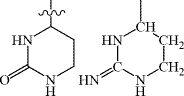 and etc. |
| R₄ | —CH₂OH | -CHO | R₄ including —CH₂NH₂, —CH═NH, —CH═NOH, —COOH, —COOR₅ (R₅ represents alkyl groups containing 1- |
| | | | 3 carbons), -CONH₂ and etc. |

Preferably, the virus refers to influenza virus and coronavirus.

The present disclosure also provides the preparation method of the peptide derivative Omicsynins, the method comprises the following steps:

following steps,
(1) The fermentation broth of Streptomyces sp. CPCC 200451 was centrifuged (4000 rpm/min, 15 min) to collect the supernatant,
(2) The active components were collected by HPLC with a reversed phase C18 column cascaded by microporous adsorption resin,
(3) The active component obtained in step (2) is separated by semi-preparative RP-HPLC to obtain the peptide derivatives,
the chemical formula of the peptide derivative Omicsynins is shown in formula (1).

The fermentation broth said in step (1) are,
transfer 10% seed to A3 medium, culture the medium at 28° C. and 200 rpm for 3-10 days, and collect the fermentation broth,
the components of the said A3 medium are: glycerol 2.0%, dextrin 2.0%, peptone 1.0%, yeast extract 0.5%, (NH₄)₂SO₄ 0.2%, CaCO₃ 0.2%. The fermentation media were adjusted to pH 7.2-7.4 using 6 mol·L-1 NaOH or 6 mol·L-1 HCl.

The step parameters of the macroporous adsorption resin cascade reverse phase HPLC method in step (2) are,
using HP20 macroporous adsorption resin and C18 reversed phase HPLC column,
and the separation steps are,
   1) the supernatant is adsorbed by macroporous adsorption resin HP20, and then washed with deionized water twice the column volume,
   2) use ethanol-water for gradient elution (20%, 50% and 100% ethanol elution in turn), each gradient elution until the effluent has no color or color unchanged, collect the eluent of 50% ethanol gradient, and use it after decompression and concentration,
   3) step 2) The obtained 50% ethanol gradient eluate is concentrated and then subjected to C18 column, and then eluted with acetonitrile-water gradient (10%, 12%, 15%, 20%, 25%, 30%, 40%, 50%, 80% and 100% acetonitrile), collecting 15%~80% gradient eluate, preferably, collecting 15%~20% acetonitrile-water eluate.

The parameters and method of semi-preparation RP-HPLC in step (3) are,
chromatographic column: SHISEIDO Capcell-Pak PFP 5 µm, 10 × 250 mm,
the mobile phases are
25% ACN/H2O containing 0.1% TFA for Omicsynin A series compounds,
20% ACN/H2O containing 0.1% HCOOH for Omicsynin B series compounds,
40% ACN/H2O containing 0.1% TFA for Omicsynin C series compounds,
flow rate: 1.5 mL/min.

The present disclosure also provides a group of peptide derivatives, Omicsynins A1, A2, A6, B1, B2, B3, B5, B6, C1, C2, C6 respectively, the general structural formula is shown in formula (1), and the substituents of R1~R4 of each compound are shown in the following table:

| | | |
|---|---|---|
| Name | R₁ | R₂ |
| Omicsynin A1 | 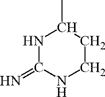 | 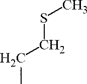 |
| Omicsynin A2 | 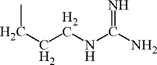 | 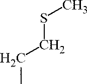 |
| Omicsynin A6 | 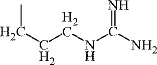 | —CH(CH₃)CH₂CH₃ Or —CH₂CH(CH₃)₂ |
| Omicsynin B1 | 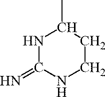 | 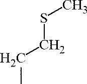 |
| Omicsynin B2 | 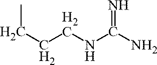 | 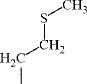 |
| Omicsynin B3 | 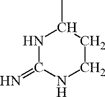 | —CH (CH₃) ₂ |
| Omicsynin B5 | 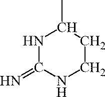 | —CH(CH₃)CH₂CH₃ Or —CH₂CH(CH₃)₂ |
| Omicsynin B6 | 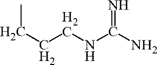 | —CH(CH₃)CH₂CH₃ Or —CH₂CH(CH₃)₂ |
| Omicsynin C1 | 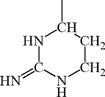 | 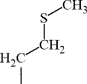 |
| Omicsynin C2 | 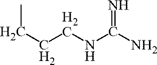 | 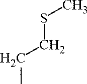 |
| Omicsynin C6 | 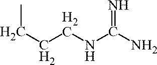 | —CH(CH₃)CH₂CH₃ Or —CH₂CH(CH₃)₂ |
| Name | R₃ | R₄ |
| Omicsynin A1 | 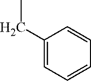 | —CH₂OH |
| Omicsynin A2 | 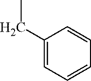 | —CH₂OH |
| Omicsynin A6 | 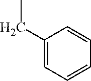 | —CH₂OH |
| Omicsynin B1 | 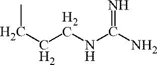 | —CHO |
| Omicsynin B2 | 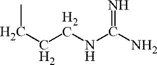 | —CHO |
| Omicsynin B3 | 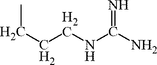 | —CHO |
| Omicsynin B5 | 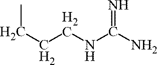 | —CHO |
| Omicsynin B6 | 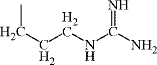 | —CHO |
| Omicsynin C1 | 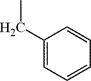 | —CHO |
| Omicsynin C2 | 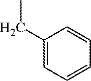 | —CHO |
| Omicsynin C6 | 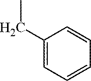 | —CHO |

The present disclosure also provides the biosynthetic gene clusters for the production of peptide derivative Omicsynins in the microorganism,
the gene cluster of Streptomyces sp. CPCC 200451 genome Chromosome 1: 7,822,964-7,875,615, with a full length of 52.6 kb,
preferably, the gene cluster between gene 7092-7102 (chromosome 1: 7,841,516-7,857,514), with a full length of 15.99 kb.

In said gene clusters, genes 7094 and 7098 are the key biosynthetic genes of Omicsynins. and the amino acid sequences of the encoded proteins are shown in SEQ ID NO.1 and 2.

The present disclosure also provides regulatory protein 7102 which are responsible for improving the expression level of the Omicsynins gene cluster. The amino acid sequence of the encoded protein is shown in SEQ ID NO.3, and its expression level is in proportion to the content of Omicsynins in the fermentation broth.

The present disclosure also provides the application of the biosynthetic gene clusters or regulatory genes in the preparation of the peptide derivative Omicsynins by microbial fermentation which is shown in formula (1).

### DESCRIPTION OF THE FIGURES

FIG. 1. Genome-wide transcriptome alignment of Streptomyces sp. CPCC 200451 under different active fermentation conditions.

FIG. 2. Transcriptome alignment at cluster 27 of Streptomyces sp. CPCC 200451 under different active fermentation conditions.

FIG. 3. Transcriptome alignment at cluster 28 of Streptomyces sp. CPCC 200451 under different active fermentation conditions.

FIG. 4. Transcriptome alignment at cluster 36 of Streptomyces sp. CPCC 200451 under different active fermentation conditions.

FIG. 5. Gene expression levels of clusters 27 in Streptomyces CPCC 200451 as detected by qRT-PCR.

FIG. 6. Gene expression levels of clusters 28 in Streptomyces CPCC 200451 as detected by qRT-PCR.

FIG. 7. Gene expression levels of clusters 36 in Streptomyces CPCC 200451 as detected by qRT-PCR.

FIG. 8. Transcriptome alignment at Cluster 36 of Streptomyces CPCC 200451 in A1, A3 and A3-Fe3+ media.

FIG. 9. Gene expression levels of clusters 36 of Streptomyces CPCC 200451 in A1, A3 and A3-Fe3+ media as detected by qRT-PCR.

FIG. 10. Comparation of the Omicsynin gene cluster and the deimino-antipain gene cluster, and their amino acid sequence identities.

FIG. 11. Biosynthetic gene clusters of Deimino-antipain and related natural products

FIG. 12. Electropherogram of plasmid pOJ7094LR by enzyme digestion

FIG. 13. Electropherogram of plasmid pOJ7098LR by enzyme digestion

FIG. 14. PCR screening of single/double crossover mutants of Streptomyces CPCC 200451

FIG. 15. PCR screening of gene 7094 double-crossover mutants of Streptomyces CPCC 200451

FIG. 16. PCR screening of gene 7098 double-crossover mutants of Streptomyces CPCC 200451

FIG. 17. The qRT-PCR analysis of gene expression levels in the core NRPS region of cluster 36 in the WT strain, 7094 gene knockout mutant (7094-KO)

FIG. 18. The qRT-PCR analysis of gene expression levels in the core NRPS region of cluster 36 in the WT strain, 7098 gene knockout mutant (7098-KO)

FIG. 19. Transcriptome alignment results of cluster 36 between the WT strain (black) and gene 7102 overexpression strain (blue, 1647/pL-7102) cultured in A1 medium

FIG. 20. The qRT-PCR analysis of gene expression levels in the core NRPS region of cluster 36 between the WT strain (black) and gene 7102 overexpression strain (blue, 1647/pL-7102) cultured in A1 medium

FIG. 21. Separation process and technical route of active compounds

FIG. 22. Chemical structures of Omicsynins A1 and A2

FIG. 23. NMR spectra for Omicsynin A1. (a) 1H-NMR in DMSO-d6, (b) 13C-NMR in DMSO-d6, (c) DEPT in DMSO-d6, (d) 1H-1H COSY in DMSO-d6, (e) HSQC in DMSO-d6, (f) HMBC in DMSO-d6, (g) 1H-1H NOESY in DMSO-d6, (h) HRMS Spectrum

FIG. 24. NMR spectra for Omicsynin A2. (a) 1H-NMR in DMSO-d6, (b) 13C-NMR in DMSO-d6, (c) DEPT in DMSO-d6, (d) 1H-1H COSY in DMSO-d6, (e) HSQC in DMSO-d6, (f) HMBC in DMSO-d6, (g) 1H-1H NOESY in DMSO-d6, (h) HRMS Spectrum

FIG. 25. Determination of the absolute configuration of Omicsynin A1 (25A-F) and Omicsynin A2 (25G-H)

FIG. 26. HRMS Spectrum of Omichin A6

FIG. 27. HRMS Spectrum of Omichin B1

FIG. 28. HRMS Spectrum of Omichin B2

FIG. 29. HRMS Spectrum of Omichin B3

FIG. 30. HRMS Spectrum of Omichin B5

FIG. 31. HRMS Spectrum of Omichin B6

FIG. 32. HRMS Spectrum of Omichin C1

FIG. 33. HRMS Spectrum of Omichin C2

FIG. 34. HRMS Spectrum of Omichin C6

FIG. 35. The effect of Omicyxin B4 on the mRNA expression level of the N protein of HCoV-OC43 in C3A cells by qRT-PCR assays.

### DETAILED DESCRIPTION OF THE EMBODIMENTS

### Materials and Methods

Strain:
Streptomyces sp. CPCC 200451 (China Pharmaceutical Culture Collection No. CPCC 200451),
Escherichia coli (E. coli) DH5a
E. coli ET12567/pUZ8002,

Plasmid:
pSET152, pSET152 derivative plasmid containing the constitutive promoter ermE*p, Amr,
pOJ260, E. coli vector, nonreplicating in Streptomyces, Amr,

Primers:

| | | | |
|---|---|---|---|
| Primer | Oligonucleotide (5′ to 3′) | Restrictio n site | Purpose |
| 7081-F | TACATATGGTTGCGGTGGTGCGGCAGGA | NdeI | Used for amplifying gene 7081 |
| 7081-R | GATCTAGACTGTTTGTTTCTGGGCGGA | XbaI | |
| 7082-F | GTACATATGGTCGTCGTGGAAATCCGGGT | NdeI | Used for amplifying gene 7082 |
| 7082-R | TAGGATCCCAACAATCTCCGACGCATCC | BamHI | |
| 7083-F | TACATATGCCCACCGACGTGTTCGGCGC | NdeI | Used for amplifying gene 7083 |
| 7083-R | TAGGATCCGAACTTCTCGCAGGTGATCG | BamHI | |
| 7089-F | TACATATGGAACTGCGGCAGCTCCAGTA | NdeI | Used for amplifying gene 7089 |
| 7089-R | GATCTAGAAGTTGACGGAGGCCAAGC | XbaI | |
| 7102-F | TATCTAGAACCGACGGCAGCTCAGAGCG | NdeI | Used for amplifying gene 7102 |
| 7102-R | TAGGATCCGCGGTGTCACCTCGTGTCCC | BamHI | |
| 7098-F | TACATATGACCACCGCGACCCTGCCCGC | NdeI | Used for amplifying gene 7098 |
| 7098-R | TAGGATCCTCATCGGGCCGCTCCCCGGG | BamHI | |
| 7098 Left arm-F | CCTAAGCTTGAGTTCCTGTACCGCTACGT | HindIII | Used for amplifying the left arm of gene 7098 |
| 7098 Left arm-F | TAGAATTCGGTGGTCCCTTCCGGGCGCC | EcoRI | |
| 7098 Right arm-F | TAGAATTCGCGCCGTGGACCAGGCCGAA | EcoRI | Used for amplifying the right arm of gene 7098 |
| 7098 Right arm-R attB- Streptomyces | CCTAAGCTTGTGCAGGCGGAGTGGATCCGGTGGGGGTGCCAGGG | HindIII - | Used for verifying the integration of ΦC31 attB site |
| pSET152 | TTCGGCGGCTTCAAGTTCGG | - | |
| 7098_KOcheckP 1 | CGAGTACCAGCACGTCCA | - | Used for verifying |
| 7098_KOcheckP 2 | GCTCGGTCTTGGTGTCGA | - | the gene 7098 knockout strain |
| 7098_KOcheckP 3 | CCCACCGACAACTTCCTGG | - | |
| 7098_KOcheckP 4 | GATCCGCTCGCAGTCCAG | - | |
| 7098_KOcheckP 5 | CTCCACCGTCCTCACCTAC | - | |
| 7098_KOcheckP 6 | CGATCCAGAGTTCGCCGA | - | |
| hrdB-RT1 | CTCATCGAGCGGGGCAAG | - | Used for the detection of hrdB transcription |
| hrdB-RT2 | CCCTCCTCAATCAGCACCTG | - | |
| 5814RT1 | GAAACTGATCCCGCGCAC | - | Used for the detection of gene 5814 transcription |
| 5814RT2 | CAGCACGCACAGGTACAC | - | |
| 5819RT1 | CTCGCCGAGGACCACATC | - | Used for the detection of gene 5819 transcription |
| 5819RT2 | GACCATCCAGAGCACCGG | - | |
| 5821RT1 | CTGCTGTTCCGGGCCTTC | - | Used for the detection of gene 5821 transcription |
| 5821RT2 | CAGGAGAGGTGTTCGGTGAG | - | |
| 7090RT1 | GGCCTCGACATCACCGTG | - | Used for the detection of gene 7090 transcription |
| 7090RT2 | CGAAGAAACGCAGGGAGAAG | - | |
| 7091RT1 | GGAAGCCAGGTCACCGAT | - | Used for the detection of gene 7091 transcription Used for the detection of gene 7092 transcription Used for the |
| 7091RT2 | CATCGGATCGCCCCAGTAG | - | |
| 7092RT1 | GGCTGATCTGGCTGTCGA | - | |
| 7092RT2 | CATGGTGGCGTTCATCCG | - | |
| 7093RT1 | CGCTACGCCTGCTGATCG | - | |
| 7093RT2 | GAGTACCCCGAGCGACTG | - | detection of gene 7093 transcription |
| 7094RT1 | ACCTGTTCACCGTCTACCG | - | Used for the detection of gene 7094 transcription |
| 7094RT2 | AGCTCCTTGGTCCAGAAGTC | - | |
| 7096RT1 | CTGGGTGATCAACGGGCG | - | Used for the detection of gene 7096 transcription |
| 7096RT2 | CTGGTCCATCGGCAGCAC | - | |
| 7097RT1 | GCACGTGATGGAGCTGTG | - | Used for the detection of gene 7097 transcription |
| 7097RT2 | AGGATGGTCGCGGTGATG | - | |
| 7098RT1 | GCCAGTCCCGGATCTTCG | - | Used for the detection of gene 7098 transcription |
| 7098RT2 | GTTGACCAGTTCGAGCCAG | - | |
| 7099RT1 | TCGACACCAAGACCGAGC | - | Used for the detection of gene 7099 transcription |
| 7099RT2 | ATGTGGTGGATCGTCAGGG | - | |
| 7101RT1 | GACGACTGGGACGCCATG | - | Used for the detection of gene 7101 transcription |
| 7101RT2 | GGGGACTTCACGACGTAGG | - | |
| 7102RT1 | GCGGCTCTTCCTCGACTAC | - | Used for the detection of gene 7102 transcription |
| 7102RT2 | CGTCCATCAGTTCGGTGAGG | - | |
| N sense | CGATGAGGCTATTCCGACTAGGT | - | Used for the detection of gene of N protein of HCoV-OC43 |
| N antisense | CCTTCCTGAGCCTTCAATATAGTAACC | - | |
| Probe-1 | TAMRA-TCCGCCTGGCACGGTACTCCCT- BHQ2 | - | |
| Sense | CGGAGTCAACGGATTTGGTCGTAT | - | Used for the |
| Antisense | AGCCTTCTCCATGGTGGTGAAGAC | - | detection of gene GAPDH |
| Probe-2 | TAMRA- CCGTCAAGGCTGAGAACGG - BHQ2 | - | |
| M2 sense | GACCRATCCTGTCACCTCTGAC | - | Used for the detection of gene of M2 protein of IAV |
| M2 antisense | GGGCATTYTGGACAAAKCGTCTACG | - | |
| β-actin sense | AGTCAAGGCTGAGAACGGGAAACT | - | Used for the detection of gene β-actin |
| β-actin antisense | TCCACAACATACTCAGCACCAGCA | - | |

### Fermentation Medium

A1 medium: glucose 0.5%, malt extract 1.0%, cottonseed powder 1.0%, soluble starch 2.0%, yeast extract 0.5%, K2HPO4 0.05%, (NH4)2SO4 0.5%, CaCO3 0.3%, NaCl 0.1%. pH 7.2-7.4.

A2 medium: glucose 0.5%, yeast extract 0.5%, peptone 0.5%, beef extract 0.5%, corn syrup 0.4%, soybean powder 1.0%, CaCO3 0.4%, CoCl2 0.002%, soluble starch 2.0% glucose 0.5%, yeast extract 0.5%, peptone 0.5%, beef extract 0.5%, corn syrup 0.4%, soybean powder 1.0%, CaCO3 0.4%, CoCl2 0.002%, soluble starch 2.0%, pH 7.2-7.4.

A3 medium: glycerol 2.0%, dextrin 2.0%, peptone 1.0%, yeast extract 0.5%, (NH4)2SO4 0.2%, CaCO3 0.2%. pH 7.2-7.4.

A4 medium: soluble starch 3.0%, soybean meal 1.5%, sodium thiosulfate 20 µ, ferrous sulfate 0.05%, dipotassium hydrogen phosphate 0.05%, potassium chloride 0.03%, pH 7.2-7.4.

B1 Glucose Asparagine Medium: Glucose 10, Aspartin 0.05%, Dipotassium Hydrogen Phosphate 0.05%, pH 7.2-7.4.

B2 Synthetic No. 5 medium: potassium nitrate 0.1%, sodium chloride 0.05%, dipotassium hydrogen phosphate 0.05%, ferrous sulfate 0.001%, magnesium sulfate 0.05%, soluble starch 2%, pH 7.0.

B3 Sodium propionate medium: sodium propionate 0.2%, ammonium nitrate 0.01%, potassium chloride 0.01%, magnesium sulfate 0.005%, ferrous sulfate 0.005%, pH 7.2.

B4 medium: sodium succinate 0.09%, ammonium dihydrogen phosphate 0.05%, magnesium sulfate 0.01%, ferrous sulfate 0.001%, pH 7.2.

B5 Waksman medium: ammonium sulfate 0.02%, dipotassium hydrogen phosphate 0.3%, magnesium sulfate 0.05%, calcium chloride 0.0126%, pH 7.2.

B6 TWYE medium: yeast powder 0.025%, dipotassium hydrogen phosphate 0.05%, pH 7.2.

B7 Kjeldahl’s synthesis medium 1: K2HPO4 0.1%, CaCO3 0.03%, NaCl 0.02%, KNO3 0.1%, FeSO4·7H2O 0.001%, CaCO3 0.05%, glucose 2.0%, pH 7.0.

B8 Chaplain medium: sucrose 3%, potassium nitrate 0.2%, dipotassium hydrogen phosphate 0.1%, potassium chloride 0.05%, magnesium sulfate 0.05%, ferrous sulfate 0.001%, pH 7.2-7.4.

B9 ISP7 medium: tyrosine 0.05%, glycerol 1.5%, asparagine 0.1%, dipotassium hydrogen phosphate 0.05%, magnesium sulfate 0.05%, sodium chloride 0.05%, ferrous sulfate 0.001%, pH 7.2-7.4.

B10 medium: soluble starch 0.2%, ferrous sulfate 0.001%, magnesium sulfate 0.05%, potassium nitrate 0.1%, sodium chloride 0.04%, dipotassium hydrogen phosphate 0.05%, pH 7.2.

Other media are conventional standardized media.

The PH of fermentation media were adjusted using 6 mol·L⁻¹ NaOH or 6 mol·L⁻¹ HCl.

### Example 1. Cultivation and Sequencing of Streptomyces CPCC 200451

### 1. Culture and Strain Preservation of Streptomyces Spp. CPCC 200451

The mycelium of CPCC 200451 was cultured in YMG or TSB liquid medium, and cultured in a shaker at 28° C. and 200 rpm for 36-72 h, when Streptomyces CPCC 200451 was cultured in solid, YMG solid medium was used at 28° C. in an incubator. Cultured for 5-7 days, MS medium was used as the solid medium for sporulation.

All strains used in the experiment were frozen in glycerol at -20° C. or -80° C.

### 2. Fermentation of Streptomyces Spp. CPCC 200451

Streptomyces sp. CPCC 200451 and its derivatives were grown at 28° C. on solid yeast malt glucose (YMG) medium for 7 days, Transfer the mycelium to a 500 mL shake flask containing 100 mL of YMG liquid medium, incubate it at 28° C. and 200 rpm for 48 h on a shaker for seed culture. Then transfer 10% seed to 500 mL shake flasks containing 100 mL of fermentation media, the fermentation broth was collected after culturing at 28° C. and 200 rpm shaker for 5 days.

TSB medium: peptone 0.2%, sodium chloride 0.5%, glucose 0.25%, dipotassium hydrogen phosphate 0.25%.

YMG medium: glucose 1%, malt extract 1%, yeast extract 1%, agar 1.5%, pH 7.0.

MS medium: mannitol 2%, soybean powder 2%, agar 2%, prepared with tap water, magnesium chloride was added at a final concentration of 10 mM at the time of use.

### 3. Genome Sequencing and Bioinformatic Analysis of Streptomyces Spp. CPCC 200451

To demystify the structure of antiviral compounds and reveal the biosynthesis mechanism, extraction of the total genomic DNA from Streptomyces sp. CPCC 200451 was carried out, and high-throughput sequencing was performed by the Beijing Genomics Institute (BGI), Shenzhen, China, with the third-generation sequencing Pacbio RSII platform and the second-generation sequencing Illumina Hiseq 4000 platform. Sequence assembly and correction were completed by SMRT Analysis v2.3.0, coupled with SOAPsnp, SOAPindel, and GATK. Glimmer 3.0 software was employed to predict protein-coding sequences (CDSs). Gene functional annotation was performed by blastp searches of the COG, KEGG, TrEMBL, Swiss-Port, NR, and GO databases. The online resource antibiotics and Secondary Metabolite Analysis Shell (antiSMASH) Version 5.0.0† was used to predict secondary metabolic biosynthesis gene clusters.

The genome of Streptomyces sp. CPCC 200451 is a linear chromosome of approximately 8,918,347 bps, with a GC content of 73.6%. The genome contains a total of 316 tandem repeat sequences with a total length of 151,923 bp, accounting for 1.7% of the total genome length. After gene annotation analysis, it was found that the genome contains 8,151 protein-coding genes, 11 rRNA operons were predicted by rRNAmmer software by aligning the rRNA library, 73 tRNA coding genes were found by tRNAscan-SE software.

### Example 2. Analysis and Confirmation of the BGC for the Biosynthesis of Antiviral Active Compounds in Streptomyces sp. CPCC 200451

### Activity-Directed Comparative Transcriptomics Analysis

The regulation of transcription level is one of the most important regulation ways in prokaryotes. In this example, comparative transcriptome analysis of fermentation broth samples with different antiviral activities (high activity, low activity, and no activity) was conducted to preliminarily locate the BGC responsible for the biosynthesis of the antiviral active compounds in Streptomyces sp. 1647.

### 1. Screening of Fermentation Conditions

We screened the fermentation conditions of CPCC 200451, tried 14 types of fermentation media and 4 fermentation time points, and determined the antivirus activity of the fermentation broth in order to obtain fermentation broth samples with different antivirus activities,

### Screening of Fermentation Medium

The spore suspension of Streptomyces CPCC 200451 was coated on the surface of YMG solid medium, and after culturing in a 28° C. incubator for 7 days, the same size of medium was picked up with an inoculation shovel and dispersed, and then inoculated into 14 different fermentation media. cultured in a shaker at 28° C. and 200 rpm.

### Selection of Fermentation Time

The fermentation broth samples of the above-mentioned different media were collected after growing for 3 days, 5 days, 7 days and 10 days, respectively, and the supernatant was collected after centrifugation to measure the antivirus activity.

### 2. Determination of Antivirus Activity of the Fermentation Samples

The determination of antivirus activity was completed by CAMS Key Laboratory of Antiviral Drug Research, Institute of Medicinal Biotechnology, Chinese Academy of Medical Sciences & Peking Union Medical College, and the virus strains were influenza virus A/ Fort Monmouth/1/47 (H1N1) and A/wuhan/359/95 (H3N2).

Steps of the antiviral assay:
(1) Use a 96-well plate to inoculate Madin-Darby canine kidney cells (MDCK), and cells seeded in plates at 37 C with 5% CO₂,
(2) After 24 hours, the cells were infected with influenza virus for 2 hours, followed by treatment with the test samples and active drugs for 48 or 72 h to set up cell control wells and virus control wells, and continue to culture,
(3) The 50% inhibitory concentration (IC50) was determined by the Reed and Muench method, The 50% toxicity concentration (TC50) of the test samples and positive compounds was also evaluated by the CPE assay. The selectivity index (SI) was calculated as the ratio of TC50/IC50.

The results of the antiviral assay showed that the fermentation broth sample from A3 medium had the highest antiviral A/wuhan/359/95 (H3N2) activity, followed by A1 fermentation medium and A2 fermentation medium. The fermentation broth samples at 5 days were significantly higher than the other 3 time points (shown in Table 1). In addition, according to the growth of the strain and the results of the antiviral assay, B7 was selected as an inactive fermentation medium as a negative control for subsequent comparative transcriptome analysis to help the screening of target biosynthetic gene clusters.

Further research showed that the fermentation broth of Streptomyces sp. CPCC 200451 from A3 medium not only had good activity against influenza virus A/wuhan/359/95 (H3N2), but also had good activity against influenza virus A/FM/1/47 (H1N1) (shown in Table 2).

TABLE 1

Antiviral activity of the fermentation crude extract from Streptomyces sp. CPCC 200451.

| | | Dose | TC₅₀ | A/wuhan/359/95(H3N2) | |
|---|---|---|---|---|---|
| Fermentation media | Fermentation time | (µg/mL or Dilution ratio) | (µg/mL or Dilution ratio) | IC₅₀ (µg/mL or Dilution ratio) | SI |
| A1 | 3d | fermentation broth⅒ | 53/10000 | 713/1000000 | 7.4 |
| A2 | 3d | fermentation broth⅒ | >⅒ | 693/10000 | >1.4 |
| A3 | 3d | fermentation broth⅒ | >⅒ | 16/10000 | >62.5 |
| A1 | 5d | fermentation broth⅒ | >⅒ | 333/10000 | >3.0 |
| A2 | 5d | fermentation broth⅒ | >⅒ | 333/10000 | 2.1 |
| A3 | 5d | fermentation broth⅒ | >⅒ | 4/10000 | >250 |
| A1 | 7d | fermentation broth⅒ | >⅒ | 192/10000 | >5.2 |
| A2 | 7d | fermentation broth⅒ | 12/10000 | 2/10000 | 6.0 |
| A3 | 7d | fermentation broth⅒ | >⅒ | 21/10000 | >47.6 |
| A1 | 10d | fermentation broth⅒ | >⅒ | 11/1000 | >9.1 |
| A2 | 10d | fermentation broth⅒ | 9/10000 | 3/10000 | 3.0 |
| A3 | 10d | fermentation broth⅒ | >⅒ | 12/10000 | >83.3 |
| Oseltamivir | | 200 | 1260 | 1.63 | 773 |

TABLE 2

Antiviral activity of the fermentation crude extract from Streptomyces sp. CPCC 200451.

| | Dose (µg/mL or Dilution ratio) | TC₅₀ (µg/mL or Dilution ratio) | A/wuhan/359/95(H3N2) | | A/Fort Monmouth/1/47(H1N1) | |
|---|---|---|---|---|---|---|
| Sample information# | | | IC₅₀ (µg/mL or Dilution ratio) | SI | IC₅₀ (µg/mL or Dilution ratio) | SI |
| A3-3d | stock solution⅒ | >⅒ | 9/100000 | >1111.1 | 8/10000 | >125.0 |
| A3-5d | stock solution⅒ | >⅒ | 7/100000 | >1428.6 | 32/10000 | >32.3 |
| Oseltamivir | 200 | 1260 | 1.63 | 773.0 | 2.01 | 626.9 |
| RBV | 200 | 1164.1 | 1.63 | 773.0 | 0.82 | 1419.6 |

### 3. Comparative Transcriptome Analysis and Real-time Quantitative PCR (RT-qPCR) Assay

According to the antiviral assay, A3 and B7 were selected as highly active and inactive fermentation media, respectively. to perform transcriptome sequencing (RNA-seq) and data analysis.

### Extraction of RNA Samples

According to the results of the antiviral assay of the fermentation broth, A3 and B7 were selected as highly active and inactive fermentation media, respectively. The mycelia and fermentation supernatants from these two media at the early stages (12 h, 24 h, 48 h) of fermentation were collected. The improved TRIzol method was used to extract the total RNA of Streptomyces CPCC 200451, and the sample names were A3-24, A3-48, A3-72, B7-24, B7-48 and B7-72.

After testing, there were 6 samples of the RNA extraction with good quality, and there was no obvious genomic DNA contamination and serious degradation, which basically met the requirements of high-throughput sequencing.

### RNA Transcriptome Sequencing and Data Analysis

RNA-sequencing (RNA-seq) was performed by the BGI (China), using the second-generation sequencing platform BGISEQ-500. the results were visualized to analyze the genome-wide transcription levels as shown in FIG. 1. In conjunction with the information of 38 secondary metabolite BGCs predicted by antiSMASH, three BGCs-namely, clusters 27, 28 and 36-showed significantly higher transcription levels under the A3 fermentation condition.

The first differentially expressed gene cluster is Cluster 27, which is an NRPS-type biosynthetic gene cluster for the production of siderophore, the results of transcription were visualized as shown in FIG. 2, It can be seen that, compared with the B7 inactive fermentation medium, Cluster 27 showed significantly high expression in the A3 highly active fermentation medium.

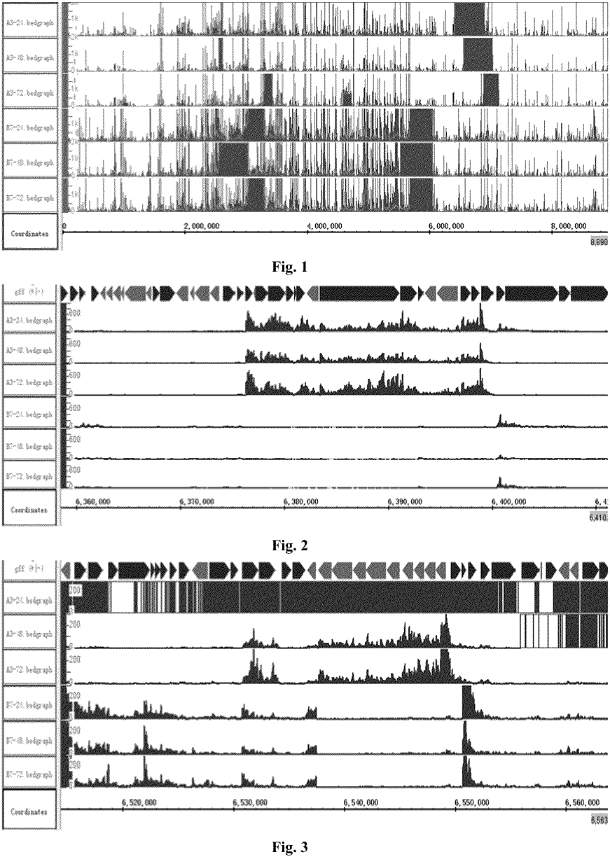

Cluster 28 is also biosynthetic gene clusters of siderophore. the results of transcription were visualized as shown in FIG. 3, It can be seen that, compared with the B7 inactive fermentation medium, Cluster 28 showed significantly high expression in the A3 highly active fermentation medium.

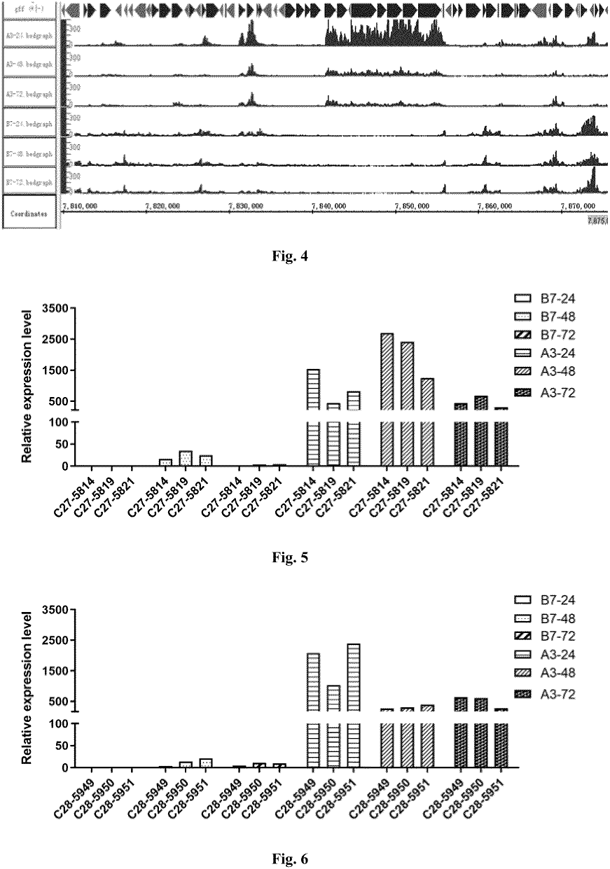

The third gene cluster showing significant differences at the transcriptional level is Cluster 36, which is an NRPS-type biosynthetic gene cluster that exhibits significantly high expression in A3 highly active fermentation medium. the results of transcription were visualized as shown in FIG. 4.

### Transcriptomics Analysis by RT-qPCR Assay

The transcriptomic results were further verified by RT-qPCR.

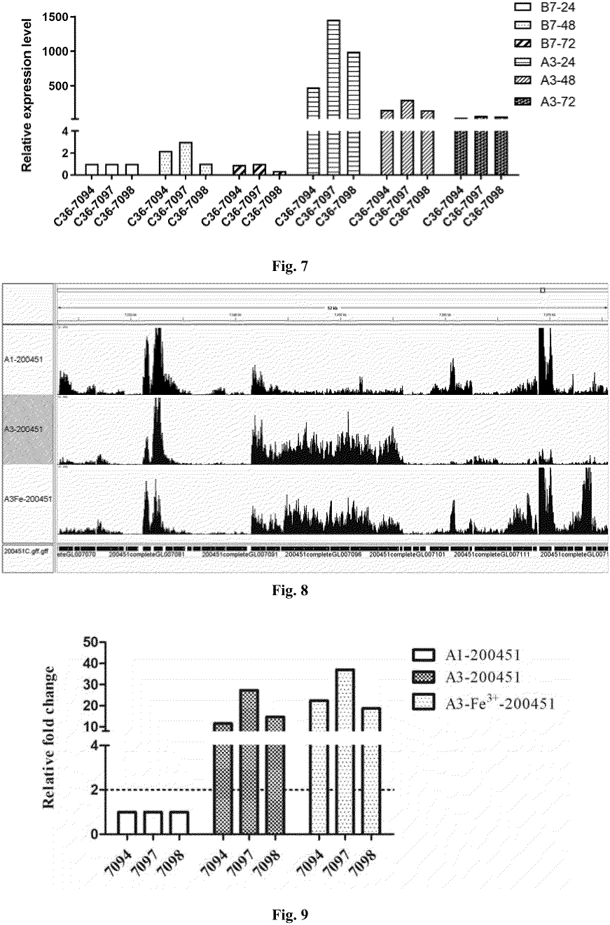

Based on bioinformatic analysis, both clusters 27 and 28 were found to be BGCs for the production of siderophores, three key genes in Cluster 27 were selected, namely C27_5814 (dhb), C27_5819 (NRPS), C27_5821 (transporter), and three key genes in Cluster 28 were selected, namely C28- 5949 (IucA_IucC), C28-5950 (transporter) and C28-5951 (ATP-grasp), Also, three functional genes, C36_7094 gene (NRPS), C36_7097 gene (NRPS) and C36_7098 gene (NRPS) in the Cluster 36 biosynthetic gene cluster were selected. the genes were subjected to RT-qPCR assay, respectively. The results are shown in FIG. 4, FIG. 5, and FIG. 7. The RT-qPCR verification results were consistent with the transcriptome sequencing results, indicating that the transcriptome sequencing data were credible.

### Example 3. Bioinformatics Analysis and Functional Analysis of Three Key Gene Clusters

Based on bioinformatic analysis, both clusters 27 and 28 were found to be BGCs for the production of siderophores, as the presence of iron box sequences in the cluster, which were closely regulated by a class of repressors in Streptomyces, sensed the presence of Fe3⁺.

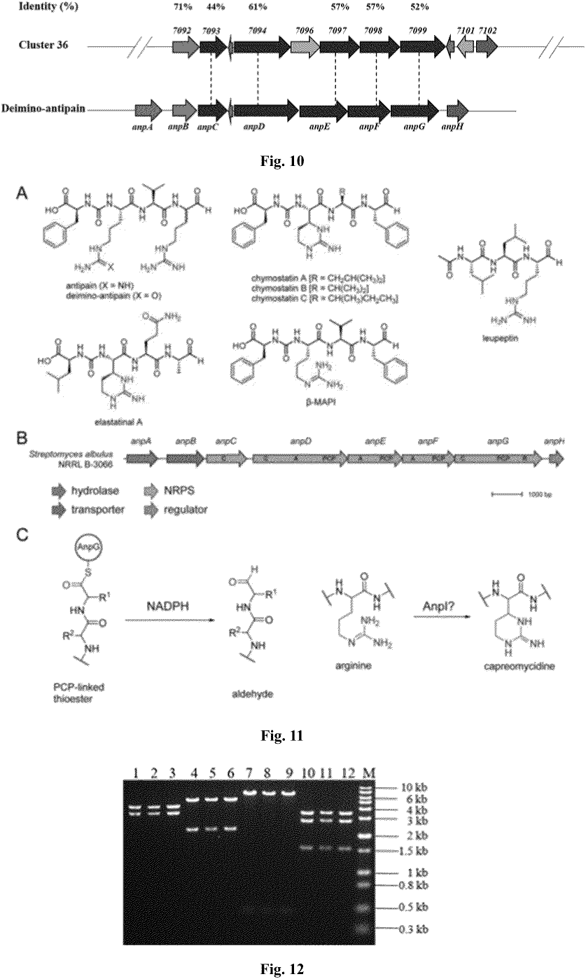

Therefore, we added 0.05% iron ions to the A3 highly active fermentation medium, namely A3-Fe³⁺ fermentation medium. Streptomyces sp. CPCC 200451 was fermented and cultured under the same conditions using both A3 and A3-Fe³⁺ media. The mycelia and fermentation supernatants from these two media at the early stages (48h) of fermentation were collected for RNA extraction, and then, RNA-Seq sequencing, data analysis, and RT-qPCR verification were performed, the transcriptome analysis demonstrated that clusters 27 and 28 were shut down after adding a sufficient amount of Fe3+ (0.05% final concentration) to the A3 fermentation medium, while cluster 36 was still highly expressed (FIG. 8). RT-qPCR was used to verify that the expression of Cluster 36 in A3 and A3-Fe³⁺ medium was significantly higher than that in A1 medium, which was consistent with the transcriptome comparison results (FIG. 9). Combined with the activity assay which showed that Streptomyces sp. CPCC 200451 fermentation broth in the presence of Fe³⁺ still exhibited a similar level of anti-IAV activity, Thus, we speculated that cluster 36 was the BGC responsible for the biosynthesis of antiviral active compounds in Streptomyces sp. CPCC 200451.

Cluster 36 is located in the genome chromosome 1: 7,822,964-7,875,615 of Streptomyces sp. CPCC 200451, with a full length of 52.6 kb. Bioinformatics analysis predicts that the gene cluster contains a total of 50 open reading frames (ORFs). The core region of the gene cluster is genes 7092-7102 (chromosome 1: 7,841,516-7,857,514).

The amino acid sequences of the proteins encoded by the 50 open reading frames contained in Cluster 36 were analyzed for homology using the BLASTP function in the GenBank database. The results showed that Cluster 36 belonged to the NRPS biosynthetic gene clusters, NRPS (Nonribosomal peptide synthetases) plays a key role in the synthesis of non-ribosomal polypeptides. It is a multifunctional protein complex composed of multiple independent modules connected in a specific spatial order. It can specifically recognize, activate and transport specific amino acid substrates which are condensed in a certain order to form peptide chains, and then, non-ribosomal polypeptides are synthesized and released. Each module in NRPS contains at least three core domains, including the adenylation domain (A domain), the peptidyl carrier protein domain (PCP domain), and the condensation domain (C domain). The last component of NRPS also contains a special domain, which is the most downstream of the synthetase peptide chain, called the thioesterase domain (TE domain), which is responsible for releasing the peptide chain from the NRPS module. In addition, other specific domains may also be included, such as epimerization domain (Epimerization, E domain), methylation domain (Methyltransferase, M domain), etc.

The biosynthetic gene clusters of Cluster 36 and Deimino-antipain have the highest similarity of 66% predicted by antiSMASH, and the similar genes are located in the core region of Cluster 36. The protein sequences encoded by the similar genes in Cluster 36 and Deimino-antipain are using BLASTP tool for comparison, the results are shown in FIG. 10. This family of protease inhibitors have been found for 40 years, they typically feature relatively low molecular weight, hydrophobicity, the presence of C-terminal aldehydes, and internal ureido linkages, and the high structural similarity suggests that they share a common biosynthesis Pathways, therefore, they have evolved to form clusters of related biosynthetic genes.

In 2016, Maxson et al. obtained Deimino-antipain from Streptomyces albulus NRRL B-3066 by a chemical reactivity-based screening, and analyzed its biosynthetic gene clusters. as shown in FIG. 11. Its BGCs are composed of the gene anpC-G NRPS, in which the A domains of the genes anpD, anpE, and anpF are responsible for the assembly of Arginine, Phenylalanine, and Valine, respectively. However, as for the fourth A domain responsible for the assembly of Arg (or Cit), one speculation is that the Phe loading module (anpE) also installs Arg/Cit, Which is similar to the biosynthesis of syringolin or the Arg specific module (anpD) in a discontinuous manner way to perform the function twice, the other case is that anpD installs Cit (or Arg is subsequently replaced by Cit) followed by Arg in a specific, non-consecutive way. The gene anpC contains the C domain only, whereas the gene anpG contains the PCP and C domains, as well as the NAD reduction (R) domain that may be responsible for the release, ultimately producing the C-terminal aldehydes. In addition, the anpA gene may encode hydrolases, which may act before or after the assembly of Arginine and play a role in the formation of Cit. The gene anpB belongs to the MFS (major facilitator superfamily) transporter superfamily, and the encoded product of anpH is histidine kinases. Subsequently, Maxson et al. demonstrated that the assembly of Cit in Deimino-antipain requires other genes by using heterologous expression.

As reported in the literatures, according to the presence of the gene anpI encoding acyl-CoA dehydrogenase and its arrangement position, when the direction and sequence of the anpB-G genes are consistent, the biosynthetic gene clusters responsible for the synthesis of such peptide aldehyde compounds roughly divided into three categories: the first category is the biosynthetic gene cluster that does not contain the gene anpI, such as Deimino-antipain, the second category is that the gene anpI is located between anpD and anpE which accounts for the majority, and only a few gene clusters have anpI located after the gene anpG, which is the third category. Therefore, Cluster 36 in Streptomyces sp. CPCC 200451 belongs to the second category. Moreover, there is an additional gene encoding SDR reductase in the gene Cluster 36, and its function needs further research.

In summary, we can speculate that the products of Cluster 36 encoded in Streptomyces sp. CPCC 200451 are rich and diverse, and the products may be structurally similar to compounds such as Deimino-antipain, chymostatin, elastatinal and MAPI. In order to further confirm whether Cluster 36 is the biosynthetic gene cluster where the antiviral components of Streptomyces CPCC 200451 are located, knockout and overexpression of genes in this strain were performed.

### Example 4. Gene Knockout Method to Verify the Function of Cluster 36 Gene Cluster in Streptomyces CPCC 200451

When CPCC 200451 was cultured in MS medium for 96-120 h, the morphology and number of spores reached the optimum state, and 120 h was selected as the spore collection time of CPCC 200451. Meanwhile, since CPCC 200451 is sensitive to apramycin, apramycin was selected as the selection marker of CPCC 200451, and Aztreonam was selected as the inhibitor of E. coli in the conjugation transfer experiment.

Establishment of CPCC 200451 Knockout Genetic Operating System:

Two NRPS-type functional genes gene 7094 (Cromosome 1: 7,844,718-7,847,825) and gene 7098 (Cromosome 1: 7,850,958-7,852,772) in Cluster 36 were selected to construct a knockout genetic operating system, respectively.

Design primers to amplify two fragments (i.e., forearm and rear arm) containing the upstream and downstream of the target genes respectively, connect them to the multiple cloning site of the suicide plasmid pOJ260, and introduce the recombinant plasmid into Streptomyces sp. CPCC 200451 by conjugation transfer. Single-crossover strains were screened using apramycin resistance marker. And then, single-crossover strains were subculture on MS solid medium without apramycin for about 5 generations, to screened for double-crossover mutant strains who lost apramycin resistance, and the results are verified by PCR technology, a blocker strain that lacks the target gene is obtained finally. The steps are as follows.

### Construction of Blocking Plasmid

Using CPCC 200451 genomic DNA as a template, two pairs of primers were designed at about 2000 bp on the left and right sides of the 7094 gene and the 7098 gene, respectively,

and the left and right homology arms for double crossover were amplified by PCR technology. The lengths of the two arms of the 7094 gene were 2129 bp and 2215 bp, respectively, the lengths of the two arms of the 7098 gene were 2056 bp and 2173 bp, respectively. HindIII and EcoRI restriction sites were introduced at both ends of the left arm, and EcoRI and HindIII restriction sites were introduced at both ends of the right arm, respectively.

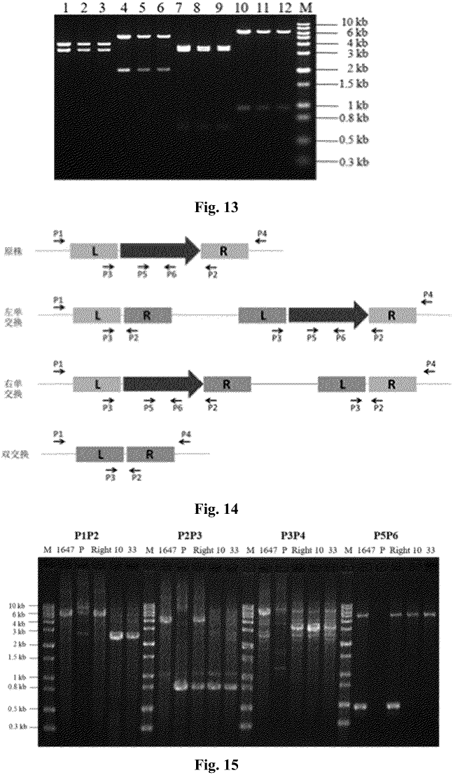

The pOJ260 suicide plasmid was selected for the construction of the blocking strain. First, the left and right homology arms obtained by PCR amplification were respectively connected to the T vector and transformed into E.coli competent cells. The recombinant plasmid was extracted for sequencing verification. and the qualified plasmid was digested with EcoRIand HindIII, the plasmid pOJ260 was digested with HindIII at the same time, and after the large fragment of the vector DNA was recovered, three fragments were ligated with the above-mentioned left and right homology arms that had been digested, and the ligated product was transformed into E. coli DH5α competent cells, The positive transformants were screened by the apramycin resistance marker located in the plasmid pOJ260 and the plasmids were extracted and verified by enzyme digestion (FIG. 12 and FIG. 13) to obtain the correct recombinant plasmids, which were named pOJ7094LR and pOJ7098LR respectively.

Lanes 1-3, pOJ7094LR/HindIII, lanes 4-6, pOJ7094LR/EcoRI, lanes 7-9, pOJ7094LR/PstI, lanes 10-12, pOJ7094LR/KpnI.

Lanes 1-3, pOJ7098LR/HindIII, lanes 4-6, pOJ7098LR/EcoRI, lanes 7-9, pOJ7098LR/PstI, lanes 10-12, pOJ7098LR/NcoI.

### Screening of Single-Crossover Mutants

The recombinant plasmids pOJ7094LR and pOJ7098LR were transformed into E. coli ET12567/pUZ8002 competent cells, and then introduced into CPCC 200451 by conjugative transfer, apramycin and aztreonam were used for resistance screening. 3-5 days later, the conjugation with apramycin resistance grew on the plate, and a single colony was picked and copied to the plate containing apramycin, which was a possible single-crossover mutant.

The single-crossover mutants were identified by PCR technology by extracting the total genomic DNA of the strains. Three pairs of primers (P1P2, P3P4 and P5P6) were designed to amplify the left homology arm and its flanking region, the right homology arm and its flanking region, and fragments of the target gene respectively (FIG. 14).

In the case of a left single crossover mutant, when PCR is performed using primer P1P2, the left homology arm except for the product fragment size of about 2 kb can be amplified. On the contrary, when using primer P3P4 to amplify the right homology arm with a product fragment size of about 2 kb, it is a right single-crossover strain.

### Screening of Double-Crossover Mutants

The qualified single-crossover strains verified by PCR were subcultured on MS solid medium without apramycin for about 5 generations, and the strains that lost apramycin resistance were screened by photocopying, and the double-crossover mutants were identified by PCR technology. When using the P3 primer located on the right edge of the left homology arm and the P2 primer located on the left edge of the right arm for PCR verification, only a small fragment of the target band can be amplified, but the fragment of the gene as long as the knockout gene cannot be amplified. the internal fragment of the gene cannot be amplified by using P5P6 primer, and the primer P1P4 can amplify the ligation product of the left homology arm and the right homology arm of about 4kb, and the 4kb PCR product is sequenced to verify, if the sequence of the left and right homology arms is correct, which proves that the strain is a double-crossover mutant.

A total of 12 single-crossover zygotes of gene 7094 were obtained, and then, one left single-crossover and one right single-crossover mutant were selected to subculture for 5 generations on MS plates without apramycin, the spores were collected for dilution and coating plate. two double-crossover mutants (named 7094-KO-10 and 7094-KO-33) were screened from the right single-crossover mutants, and verified by PCR, as shown in FIG. 15.

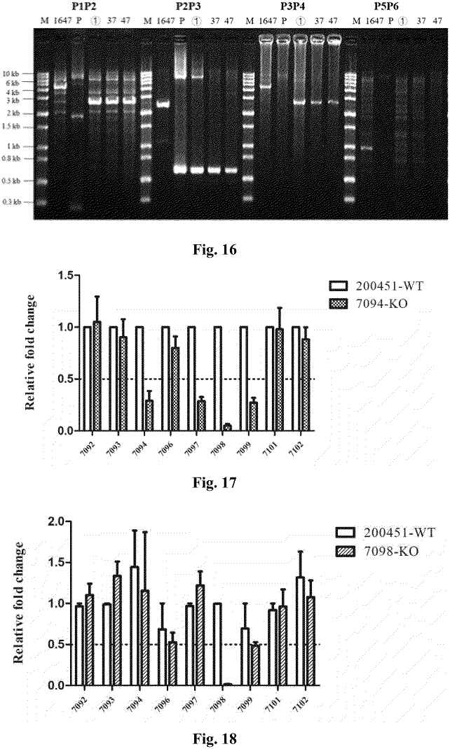

The 7098 gene was screened on a plate containing apramycin resistance, and only one zygote was obtained, which was verified by PCR. Similarly, after subculturing this strain on MS plates without apramycin for 5 generations, we screened for double-crossover mutants that lost apramycin resistance (named 7098-KO-37 and 7098- KO-47), and PCR identification was performed, as shown in FIG. 16.

### Validation of Blocking Strains by RT-qPCR

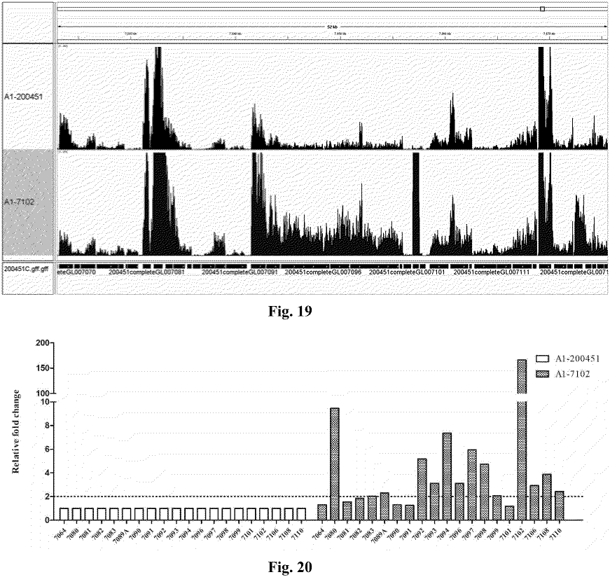

The blocking strains 7094-KO, 7098-KO and CPCC 200451 wild-type strains obtained by the above screening were fermented under the same conditions using A3-Fe³⁺ medium with excess iron ions. the mycelia were collected at the early stages of fermentation (48 h) and RNA was extracted, after reverse transcribing into cDNA, RT-qPCR was performed to verify the related genes of Cluster 36 (FIGS. 17 and 18). The results showed that the target gene had been successfully knocked out, at the same time, it was also found that when the 7094 gene was knocked out, the 7098 gene was no longer expressed, and the expression of the 7097 gene and the 7099 gene was also affected, and after the 7098 gene was knocked out, the 7094 Gene expression was not affected.

### Construction of Complementary Strains

Using the genomic DNA of wild-type Streptomyces sp. CPCC 200451 as the template, primers 7094_F (containing NdeI restriction site), 7094_R (containing XbaI restriction site), 7098_F (containing NdeI restriction site) and 7098_R (containing NdeI restriction site) were designed respectively. The 7094 gene and the 7098 gene were amplified by PCR technology and cloned into the pSET152 plasmid (containing a strong erythromycin promoter and phage ΦC31 integration site, with apramycin resistance), 7094 gene and 7098 gene genetically complementary recombinant plasmids were constructed respectively on the corresponding restriction sites. After verification by enzyme digestion and sequencing, the complementary plasmids pL-7094 and pL-7098 were obtained.

The correct complementing plasmids were introduced into the blocking strains 7094-KO and 7098-KO by conjugation transfer, and the apramycin resistance of the plasmid pSET152 was used as the selection marker, and three zygotes were selected for each gene to be verified by PCR technology using 3 pairs of primers including apramycin resistance, pSET152 integration site and apoplexy gene. The results showed that the genetically complementary strains 7094-KOC and 7098-KOC were successfully constructed.

### Validation of Strains by RT-qPCR

The blocking strains 7094-KO and 7098-KO obtained, the complementary strains 7094-KOC, 7098-KOC and the Streptomyces sp. CPCC 200451 wild-type strain were simultaneously fermented under the same conditions using A3-Fe3+ fermentation medium.

the mycelia were collected at the early stages of fermentation (48 h) and RNA was extracted, after reverse transcribing into cDNA, RT-qPCR was performed to verify the related genes. The results showed that the 7094 gene was successfully complemented into the knockout strain 7094-KO, the 7098 gene was successfully complemented into the blocker strain 7098-KO.

### Determination of Anti-Influenza Virus Activity of Blocking Strains and Complementary Strains

The anti-influenza virus activity of the fermentation broths of the blocking strains 7094-KO, 7098-KO, the complementing strains 7094-KOC, 7098-KOC and the CPCC 200451 wild-type strains was tested.

The results showed that knockout of the gene 7094 Or complementation of this gene, the anti-influenza virus activity of Streptomyces CPCC 200451 disappeared, while inactivation of gene 7098 led to a loss of anti-influenza virus activity, and complementation of this gene could restore the antiviral activity (Table 3). Therefore, it was proved that the expression of Cluster 36 was closely related to the anti-influenza virus activity of Streptomyces sp. CPCC 200451.

TABLE 3

Determination of the anti-influenza virus activity of the fermentation samples of the strains WT, blocking strain, and complementing strain cultured by A3 + Fe³⁺ medium.

| Sample information | Dose (µg/mL or dilution ratio) | TC₅₀ (µg/mL or dilution ratio) | A/Wuhan/359/1995 (H3N2) | |
|---|---|---|---|---|
| | | | IC₅₀ (µg/mL or dilution ratio) | SI |
| 200451-WT | stock solution⅒ | >⅒ | 16/10000 | >62.5 |
| 7094-KO-10 | stock solution⅒ | >⅒ | >1/30 | - |
| 7094-KO-33 | stock solution⅒ | >⅒ | >1/30 | - |
| 7094-KOC-1 | stock solution⅒ | >⅒ | >⅒ | - |
| 7094-KOC-2 | stock solution⅒ | >⅒ | >⅒ | - |
| 7094-KOC-3 | stock solution⅒ | >⅒ | >⅒ | - |
| 7098-KO-37 | stock solution⅒ | >⅒ | 33/1000 | >3.0 |
| 7098-KO-47 | stock solution⅒ | >⅒ | >1/30 | - |
| 7098-KOC-1 | stock solution⅒ | >⅒ | 26/10000 | >38.5 |
| 7098-KOC-2 | stock solution⅒ | >⅒ | 48/10000 | >20.8 |
| 7098-KOC-3 | stock solution⅒ | >⅒ | 37/10000 | >27.0 |
| Oseltamivir | 1000 | 577.4 | 2.01 | 287.3 |

### Example 5. Establishment of Overexpression Genetic Operating System

To further confirm the role of cluster 36 in the biosynthesis of the antiviral metabolites in Streptomyces sp. CPCC 200451, we selected 5 regulatory genes in this gene cluster, constructed overexpression plasmids based on plasmid pSET152 and introduced them into Streptomyces sp. CPCC 200451. The recombinant strains were fermented under the same conditions using A1 fermentation medium, and by detecting and comparing the changes in anti-influenza virus activity, the regulatory genes and their regulatory effects on the expression of anti-influenza active ingredients in Cluster 36 were determined.

### 1. Construction of Regulatory Gene Overexpression Plasmid

First, using the genomic DNA of Streptomyces sp. CPCC 200451 as a template, five regulatory genes 7081, 7082, 7083, 7089 and 7102 in Cluster 36 were selected, and primers were designed respectively to amplify the DNA fragments of these five regulatory genes by using PCR technology.

The integrated plasmid pSET152 was digested with NdeI and BamHI. Since the regulatory genes 7081 and 7089 contain BamHI sites, NdeI and XbaI sites were introduced at their two ends respectively. The plasmid pSET152 was digested with NdeI and XbaI at the same time. NdeI and BamHIrestriction sites were introduced into both ends of 7082, 7083 and 7102 regulatory genes.

the PCR product was ligated to the pEASY-T vector, and after sequencing to verify that the sequence was correct, the regulatory gene was digested with the corresponding restriction site and the product was recovered, and then it was connected to the plasmid pSET152 vector after the same restriction enzyme digestion. to obtain recombinant plasmids.

### 2. Introducing the Recombinant Plasmids Into Streptomyces Spp. CPCC 200451 By Electrotransformation

The above recombinant plasmids were introduced into Streptomyces sp. CPCC 200451 wild-type strain by electrotransformation, and screened by apramycin resistance to obtain overexpressed recombinant strains. At the same time, the empty vector pSET152 was introduced into the wild-type strain of Streptomyces sp. CPCC 200451 as a control. The primers for PCR verification are pSET152 and attB-Streptomyces. If the recombinant plasmid is correctly integrated into the genome of Streptomyces sp. CPCC 200451, the target band of 1.6kb can be amplified by PCR. The result showed that regulated gene 7081, 7082, 7083 and 7102 obtained 3 overexpressing strains respectively, regulated gene 7089 obtained 2 recombinant strains, named 200451/pL-7081, 200451/pL-7082, 200451/pL-7083, 200451/pL-7089 and 200451/pL-7102 respectively.

### 3. Determination of Anti-Influenza Virus Activity of Overexpressed Strains

In order to further explore the effect of up-regulation of regulatory genes in Cluster 36 on the anti-influenza virus activity of Streptomyces sp. CPCC 200451, three fermentation media, A1, A3 and B7, were selected to ferment the overexpressed strains on the same conditions. and the fermentation broth samples were collected for the determination of anti-influenza virus activity (Table 4). The results showed that when the regulatory gene 7102 was overexpressed, the anti-influenza virus activity of the fermentation samples from A1 and A3 medium showed a certain degree of improvement, after overexpressing the other four regulatory genes, the antiviral activity of the fermentation broth was not obvious. In addition, the fermentation products using B7 medium did not show significantly improved anti-influenza virus activity, mainly because B7 is an oligotrophic medium, and the growth of streptomyces is restricted to synthesize abundant secondary grade metabolites.

TABLE 4

Determination of anti-influenza virus activity of overexpressed strains

| Strain information | Fermentation media | Dose (µg/mL or dilution ratio) | TC₅₀ (µg/mL or dilution ratio) | A/Wuhan/359/1995 (H3N2) | |
|---|---|---|---|---|---|
| | | | | IC₅₀ (µg/mL or dilution ratio) | SI |
| 200451-WT | A1 | stock solution⅒ | >⅒ | 104/10000 | >9.6 |
| 200451/pL-7102 | A1 | stock solution⅒ | >⅒ | 39/10000 | >25.6 |
| 200451-WT | A3 | stock solution⅒ | >⅒ | 21/10000 | >47.6 |
| 200451/pL-7102 | A3 | stock solution⅒ | >⅒ | 6/10000 | >166.8 |
| 200451-WT | B7 | stock solution⅒ | >⅒ | >⅒ | - |
| 200451/pL-7102 | B7 | stock solution⅒ | >⅒ | 50/1000 | >2.0 |
| Oseltamivir | | 1000 | >1000 | 1.77 | >565.0 |

### 4. Transcriptome Analysis of Overexpression Strains

he overexpression of gene 7102 caused an increase in the anti-IAV activity of the fermentation samples of A1 medium, we conducted a further study on the difference in the transcription level of this change. the wild-type (WT) strain and gene 7102 overexpression strain were simultaneously fermented by using A1 fermentation medium, and the mycelia were collected at the early stages of fermentation (48 h) and RNA was extracted to perform transcriptome sequencing (RNA-Seq) and data analysis. The transcription of Cluster 36 was visualized using a visualization tool (FIG. 19). The results showed that the genes in the core region of Cluster 36 (Chromosome 1: 7,841,516-7,858,166) were significantly up-regulated after the overexpression of the regulatory gene 7102.

### 5. Validation of Overexpression Strains by RT-qPCR

In order to verify the reliability of the transcriptome data, we also reverse transcribed the extracted RNA samples into cDNA for quantitative RT-qPCR verification. The results are shown in FIG. 20. Compared with the wild-type strain of Streptomyces sp. CPCC 200451, the mycelia collected from the A1 fermentation medium overexpressed the regulatory gene 7102, and the genes in the core region of Cluster 36 showed a 2-8-fold increase. This change is consistent with the transcriptome results.

Therefore, we infer that the regulatory gene 7102 regulates the expression of genes in the core region of Cluster 36, and the up-regulation of gene 7102 will cause the increase of the anti-influenza virus activity of Streptomyces sp. CPCC 200451, that is, The positive regulatory function of the genes 7102 is confirmed.

To sum up, this study selected two functional genes and constructed knockout genetic operating systems (7098-ko, 7094-ko). The results of RT-qPCR verification showed that the two functional genes were successfully knocked out, and the knockout strains and wild-type of Streptomyces CPCC 200451 were fermented and tested for their anti-influenza virus activity. The results showed that inactivation of the functional genes led to a loss of anti-influenza virus activity. we also performed overexpression of multiple regulatory genes situated in this cluster. The five regulatory genes-namely, 7081 (TetR family), 7082 (streptomyces antibiotic regulatory protein (SARP) family), 7083 (ArsR family), 7089 (LysR family), and 7102 (TetR family) were selected, the results revealed that only the overexpression of gene 7102 caused an increase in the anti-IAV activity. The gene expression changes of cluster 36 between the wild-type (WT) strain and gene 7102 overexpression strain shown by the RNA-seq data suggested that overexpression of gene 7102 did indeed cause a significant up-regulation of the genes in the NRPS core region of cluster 36. This finding was confirmed by qRT-PCR. Therefore, cluster 36 was the BGC responsible for the biosynthesis of antiviral active compounds in Streptomyces sp. CPCC 200451.

### Example 6. Chemical Separation and Purification of Antiviral Components

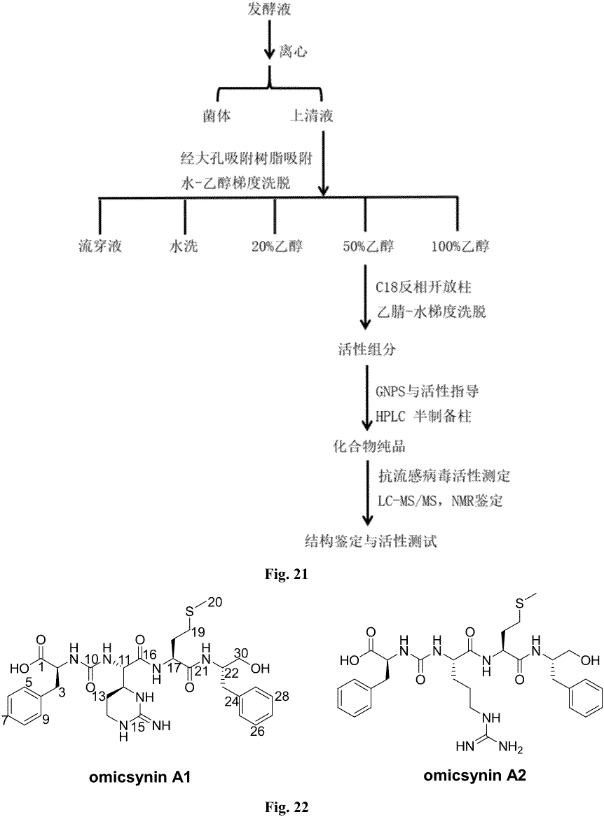

It showed that adding excess iron ions to A3 highly active medium can make the biosynthesis gene cluster of siderophore no longer expressed while Cluster 36 can be highly expressed in the preliminary experiments. Therefore, the Streptomyces sp. CPCC 200451 strain was fermented in large quantities by using A3-Fe³⁺ medium, At the same time, the knockout mutants 7098-KO and 7094-KO as a negative control was fermented under the same conditions, and the supernatant of the fermentation was collected by centrifugation. Guided by the results of HPLC analysis and antiviral assay, the components with anti-influenza virus activity were traced, and the samples were prepared and purified by HPLC. The separation process of active compounds is shown in FIG. 21.

The fermentation broth of the wild-type stain of Streptomyces sp. CPCC 200451 was centrifuged to collect a total of 14 L of the supernatant, The supernatant was adsorbed by microporous adsorption resin (Diaion HP20, Mitsubishi, Japan). The column was then rinsed with twice the column volume of deionized water. Gradient elution was carried out with 20%, 50%, and 100% ethanol-water (v/v), respectively. Each gradient was eluted until the effluent had no color and designated as crude extracts 20E, 50E, and 100E, respectively. The eluates of each gradient were separately collected, condensed by evaporation and lyophilized to determine their anti-influenza virus activity, The results showed that the antiviral active components were mainly concentrated in 50E elution, in addition, the 100E eluted fraction also had some activity (Table 5).

TABLE 5

The results of anti-influenza virus activity assay

| Sample NO. | Dose (µg/mL or dilution ratio) | TC₅₀ (µg/mL or dilution ratio) | A/Wuhan/359/1995 (H3N2) | |
|---|---|---|---|---|
| | | | IC₅₀ (µg/mL or dilution ratio) | SI |
| 200451 | stock solution | 577/10000 | 21/10000 | 27.5 |
| 200451-unadsorbed fraction | stock solution⅒ | >⅒ | >1/30 | - |
| 200451-water eluent | stock solution⅒ | >⅒ | >1/30 | - |
| 200451-20E | 500 | >500 | >500 | - |
| 200451-50E | 500 | >500 | 10.69 | >46.8 |
| 200451-100E | 500 | >500 | 23.86 | >21.0 |
| 7098-KO | stock solution⅒ | ⅒ | >⅒ | - |
| 7094-KO-20E | 500 | >500 | >166.67 | - |
| 7094-KO-50E | 500 | >500 | >166.67 | - |
| 7094-KO-100E | 500 | 288.68 | 166.67 | 1.7 |
| 7098-KO | stock solution⅒ | 577/10000 | 33/1000 | 1.7 |
| 7098-KO-20E | 500 | >500 | >500 | - |
| 7098-KO-50E | 500 | >500 | >500 | - |
| 7098-KO-100E | 500 | 288.68 | >166.67 | - |
| Oseltamivir Phosphate | 1000 | 577.35 | 2.01 | 287.3 |
| RBV | 1000 | >1000 | 2.47 | >404.9 |

The 50E eluent with the best anti-influenza virus activity obtained was concentrated and then Octadecylsilyl silica gel (ODS-A-HG, YMC, Japan) was used for the open column chromatography to conduct further separation. Gradient elution was carried out with 10%, 12%, 15%, 20%, 25%, 30%, 40%, 50%, 80% and 100% acetonitrile-water (v/v), each elution were analyzed by HPLC using an Agilent-C18-Aq analytical column (5 µm, 4.6 × 150 mm) with acetonitrile and water (containing 0.1% TFA) as mobile phase, analysis conditions are 0-30 min (0-30% acetonitrile), 30-60 min (30-100% acetonitrile). According to the HPLC analysis results of the main components in each fraction, a total of 10 components (A-J) were obtained after merging, which were expressed as 50E-C18-A~J, respectively, the 10 components were tested for anti-influenza virus activity (shown in Table 6). The results showed that the 50E-C18-C~I components all had certain anti-influenza virus activity, and the antiviral activities of the 50E-C18-E and 50E-C18-F components were significantly higher than those of the other components.

TABLE 6

Determination of anti-influenza virus activity of components in 50E-C18

| Sample information | Dose (µg/mL or dilution ratio) | TC50 (µg/mL or dilution ratio) | A/Wuhan/359/1995 (H3N2) | |
|---|---|---|---|---|
| | | | IC₅₀ (µg/mL or dilution ratio) | SI |
| 50E-C18-A | 500 | >500 | >500 | - |
| 50E-C18-B | 250 | >250 | 44.92 | >5.6 |
| 50E-C18-C | 500 | >500 | 6.17 | >81.0 |
| 50E-C18-D | 250 | >250 | 4.99 | >50.1 |
| 50E-C18-E | 500 | >500 | 2.06 | >242.7 |
| 50E-C18-F | 500 | >500 | 4.09 | >122.2 |
| 50E-C18-G | 500 | >500 | 12.35 | >40.5 |
| 50E-C18-H | 500 | >500 | 166.67 | >3.0 |
| 50E-C18-I | 250 | >250 | 12.19 | >20.5 |
| 50E-C18-J | stock solution⅒ | >⅒ | 84/10000 | >11.9 |
| Oseltamivir | 1000 | 577.35 | 2.47 | 233.7 |
| RBV | 1000 | >1000 | 2.47 | >404.9 |

### Example 7. Isolation and Structural Identification of Secondary Metabolites

Omicsynin A was isolated from 50E-C18-G component by RP-HPLC (SHISEIDO Capcell-Pak PFP 5 µm, 10 × 250 mm, 25% ACN/H2O containing 0.1% TFA, 1.5 mL/min),

The components 50E-C18-E and 50E-C18-F were directly semi-prepared by RP-HPLC (SHISEIDO Capcell-Pak PFP 5 µm, 10 × 250 mm, 20% ACN/H2O with 0.1% HCOOH, 1.5 mL/min) to obtain a group of compounds, named Omicynin B.

The component 200451-100E was directly semi-prepared by RP-HPLC (SHISEIDO Capcell-Pak PFP 5 µm, 10×250 mm, 40% ACN/H2O containing 0.1% TFA, 1.5 mL/min) to obtain a group of compounds, named as Omicsynin C.

the chemical structures of the compounds were deduced by analysis of spectroscopic data including HRESIMS, ¹H-NMR, ¹³C-NMR, DEPT, 1H-1H COSY, HSQC, HMBC and NOESY. as shown in Table 7 below.

TABLE 7

Structure identification of Omichin A-C

| | | |
|---|---|---|
| Description | Structural Formula | Structural Identification |
| Omicsynin A1 | 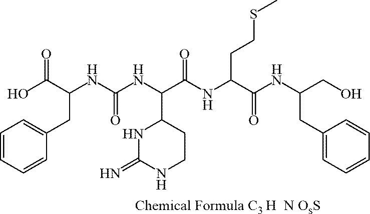 | NMR HRMS |
| Omicsynin A2 | 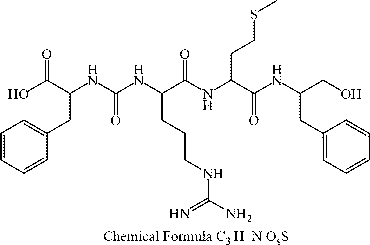 | NMR HRMS |
| Omicsynin A3 (Chymostatinol A) | 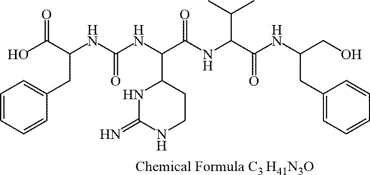 | NMR HRMS |
| Omicsynin A4 | 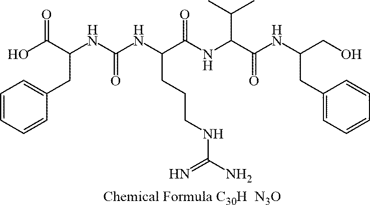 | NMR HRMS |
| Omicsynin A5 (Chymostatinol B or C) [R═CH(CH₃)CH₂CH₃ or CH₂CH(CH₃)₂] | 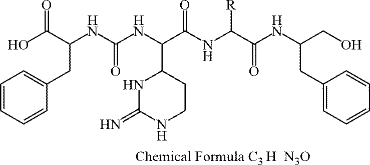 | HRMS |
| Omicsynin A6 [R═CH(CH₃)CH₂CH₃ or CH₂CH(CH₃)₂] | 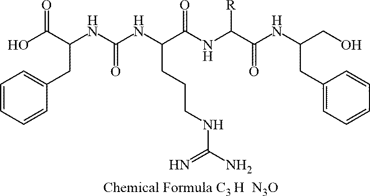 | HRMS |
| Omicsynin B1 | 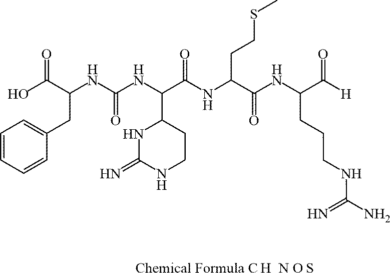 | HRMS |
| Omicsynin B2 | 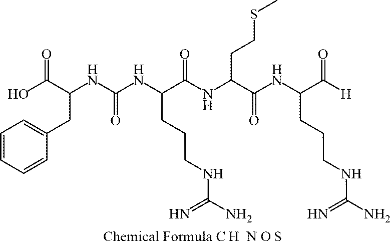 | HRMS |
| Omicsynin B3 | 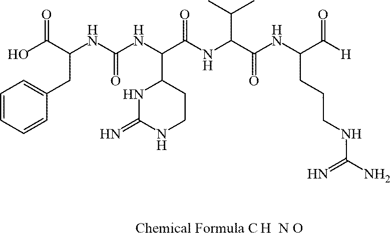 | HRMS |
| Omicsynin B4 (Antipain) | 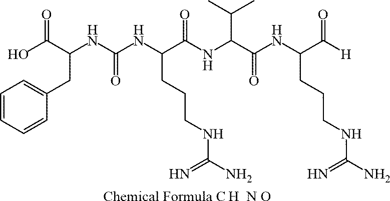 | HRMS |
| Omicsynin B5 [R═CH(CH₃)CH₂CH₃ or CH₂CH(CH₃)₂] | 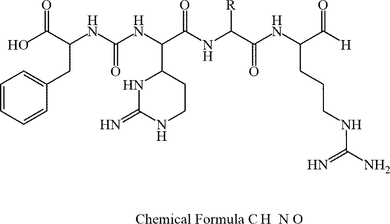 | HRMS |
| Omicsynin B6 [R═CH(CH₃)CH₂CH₃ or CH₂CH(CH₃)₂] | 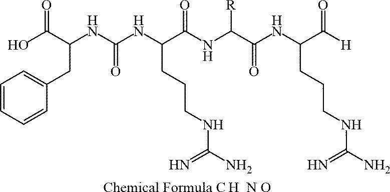 | HRMS |
| Omicsynin C1 | 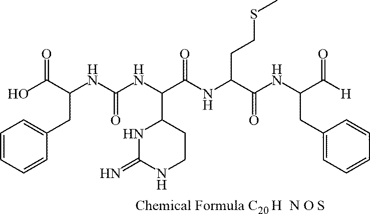 | HRMS |
| Omicsynin C2 | 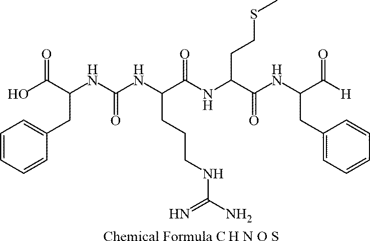 | HRMS |
| Omicsynin C3 (Chymostatin B) | 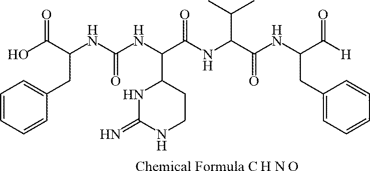 | HRMS |
| Omicsynin C4 | 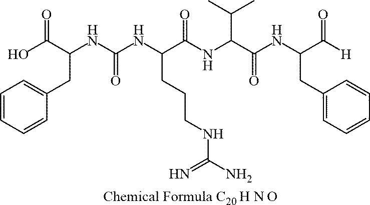 | HRMS |
| Omicsynin C5 (Chymostatin A or C) [R═CH₂CH(CH₃)₂ or CH(CH₃)CH₂CH₃] | 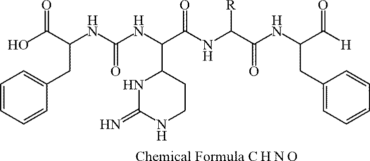 | HRMS |
| Omicsynin C6 [R═CH₂CH(CH₃)₂ or CH(CH₃)CH₂CH₃] | 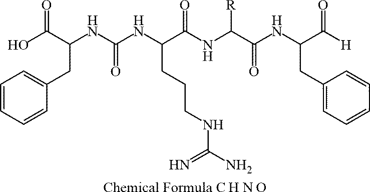 | HRMS |

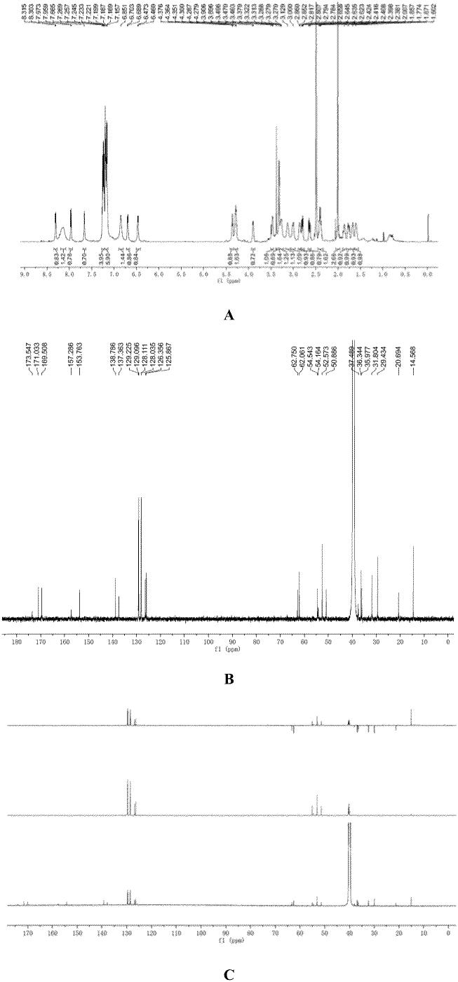

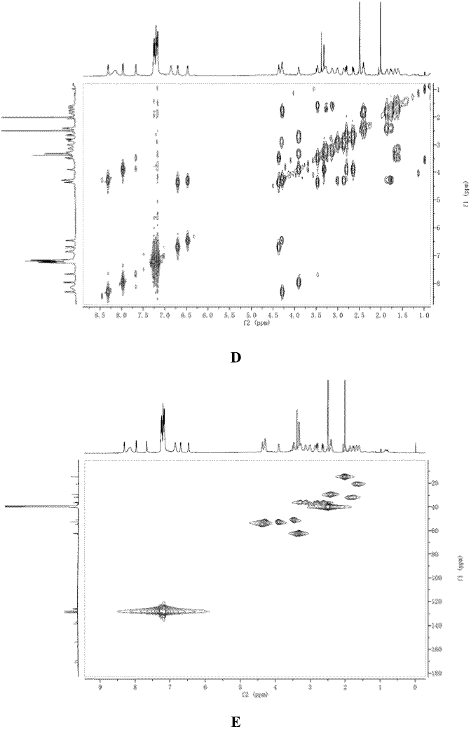

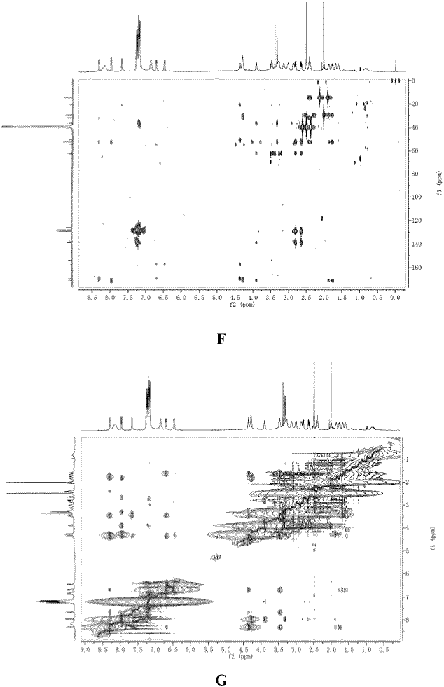

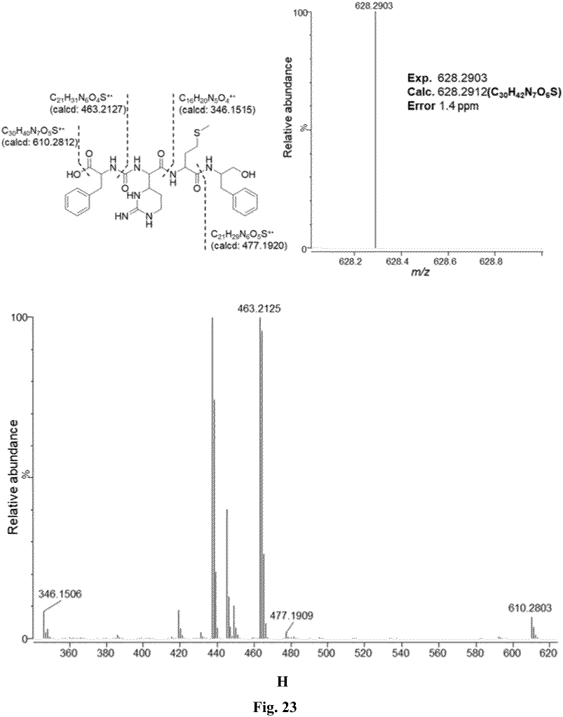

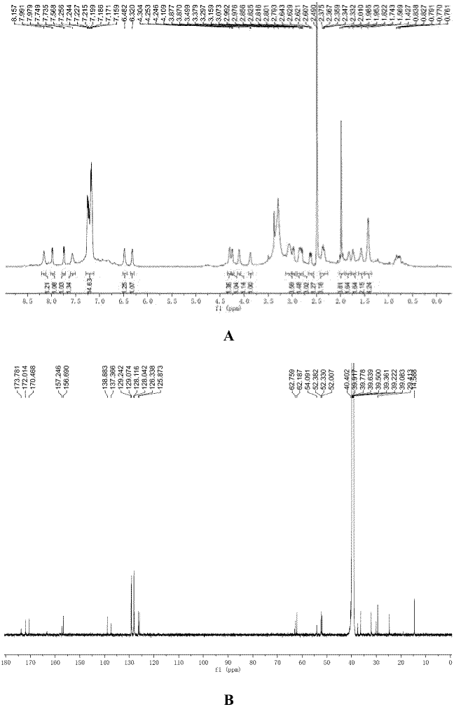

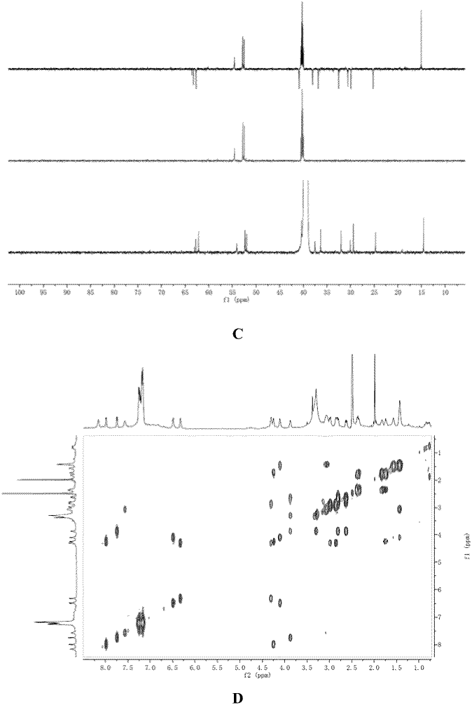

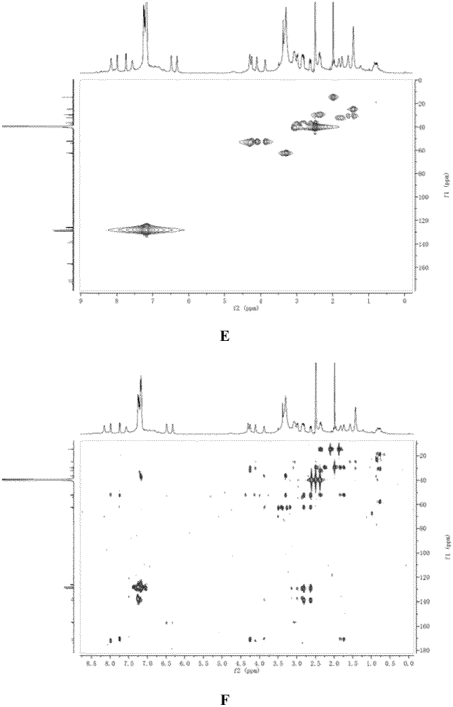

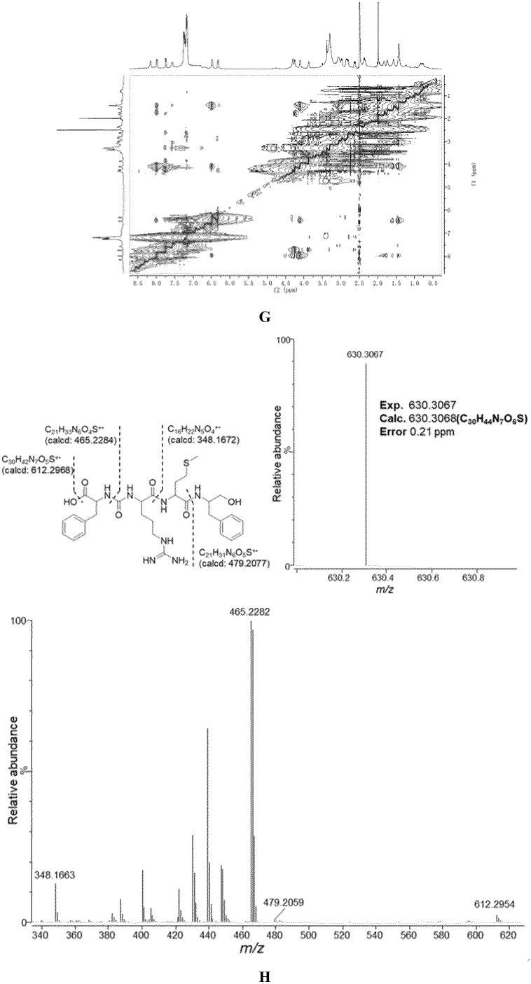

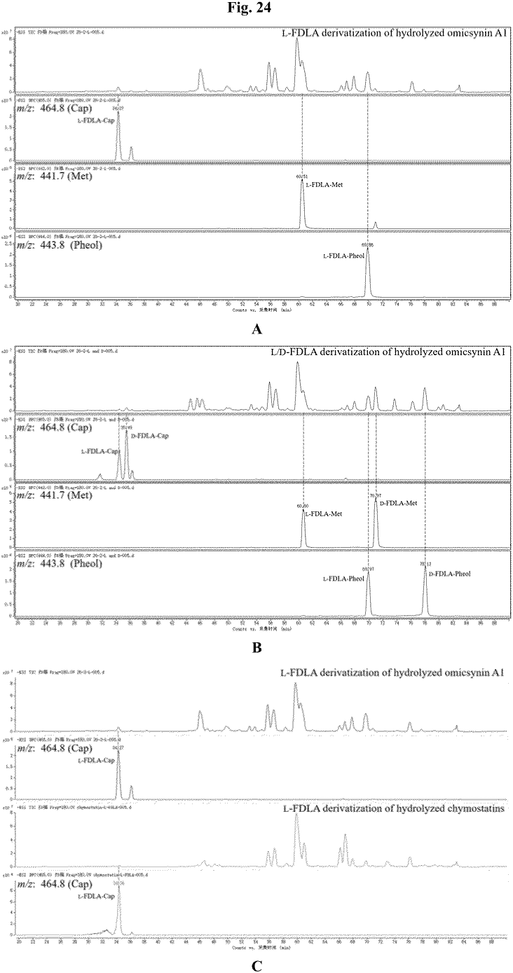

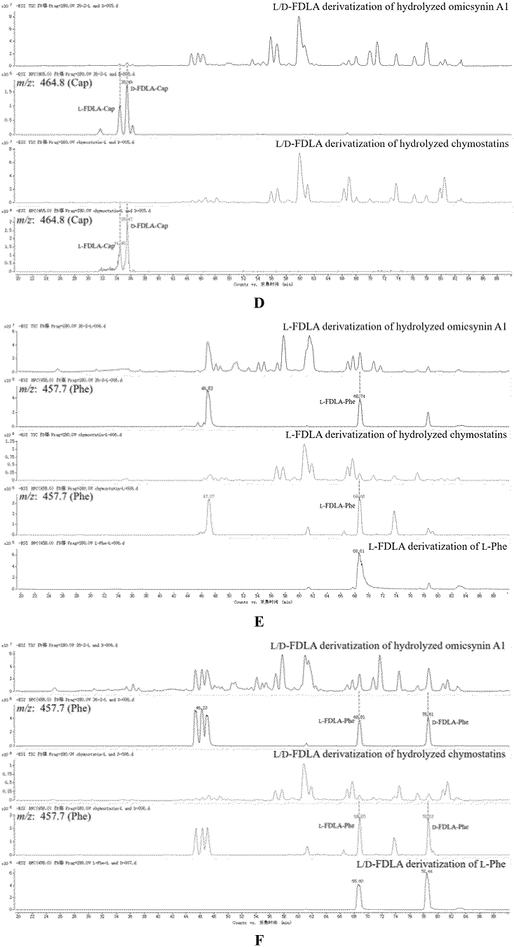

Omicsynin A1 and Omicsynin A2 are new compounds as shown in FIG. 22, and the NMR data are shown in Table 8. Data of 1H-NMR, 13C-NMR, DEPT, 1H-1H COSY, HSQC, HMBC, NOESY spectra and HRMS analysis data are shown in FIGS. 23 and 24.

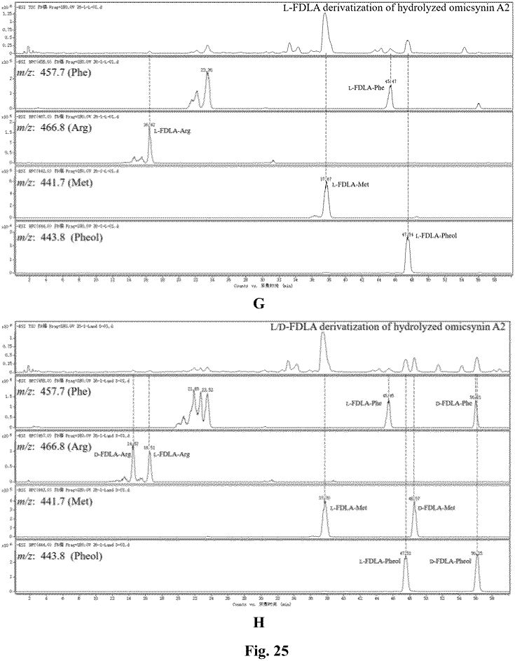

The absolute configuration of Omicsynin A1 and Omicsynin A2 was determined by Marfey method, as shown in FIG. 25.

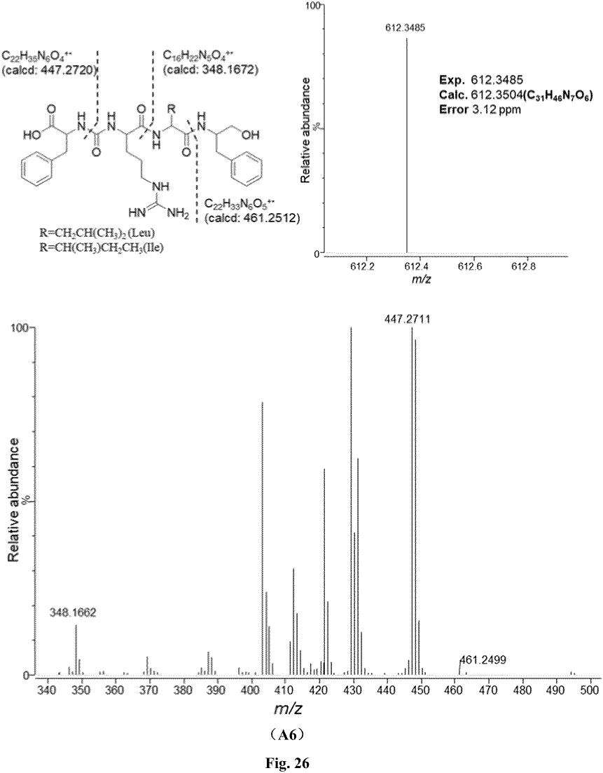

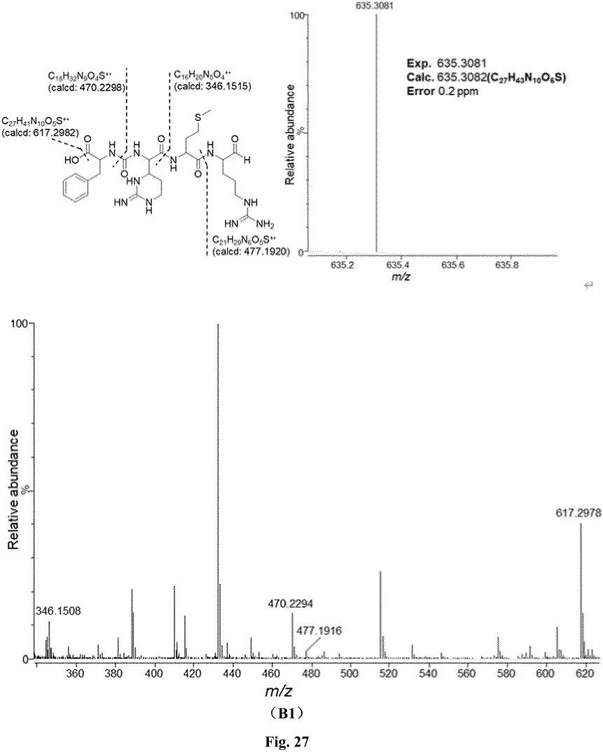

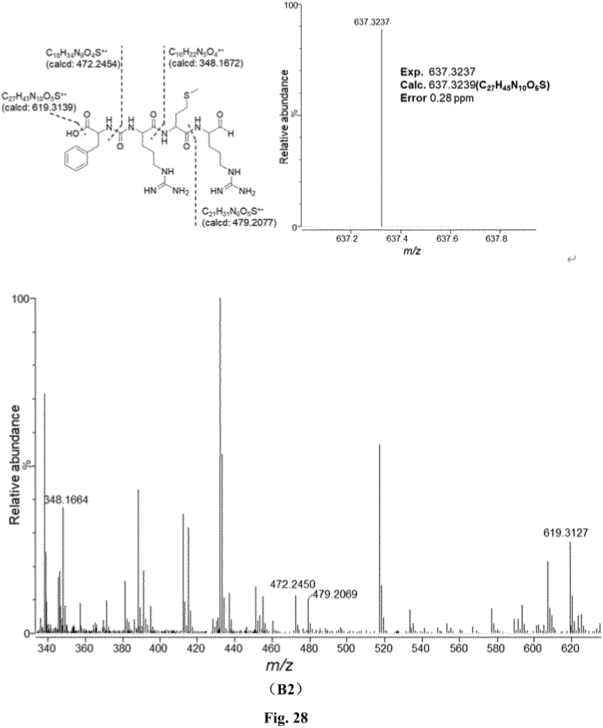

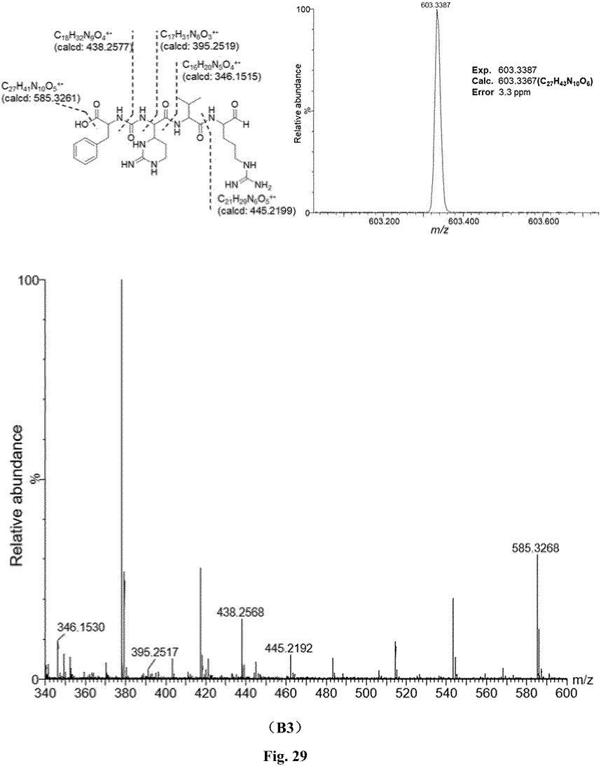

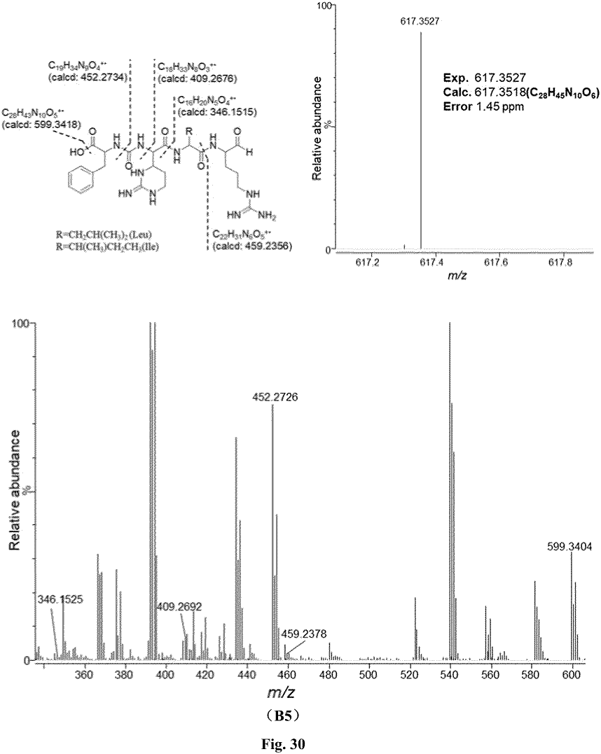

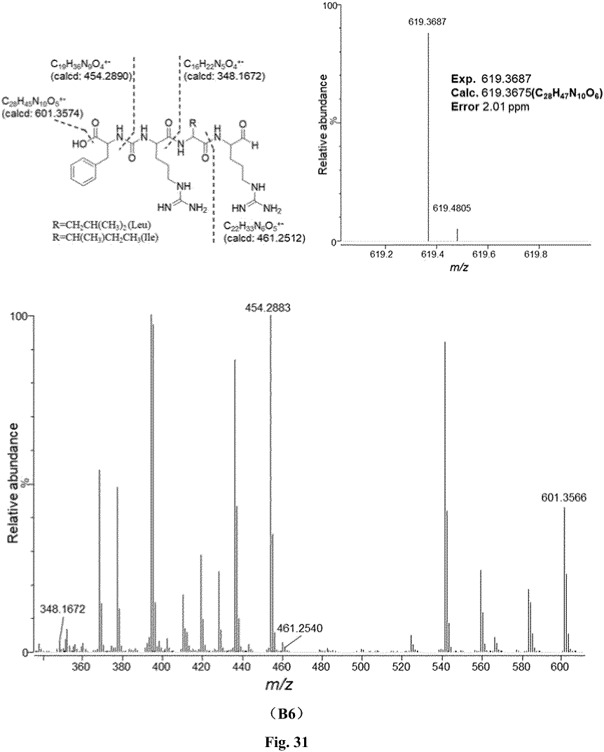

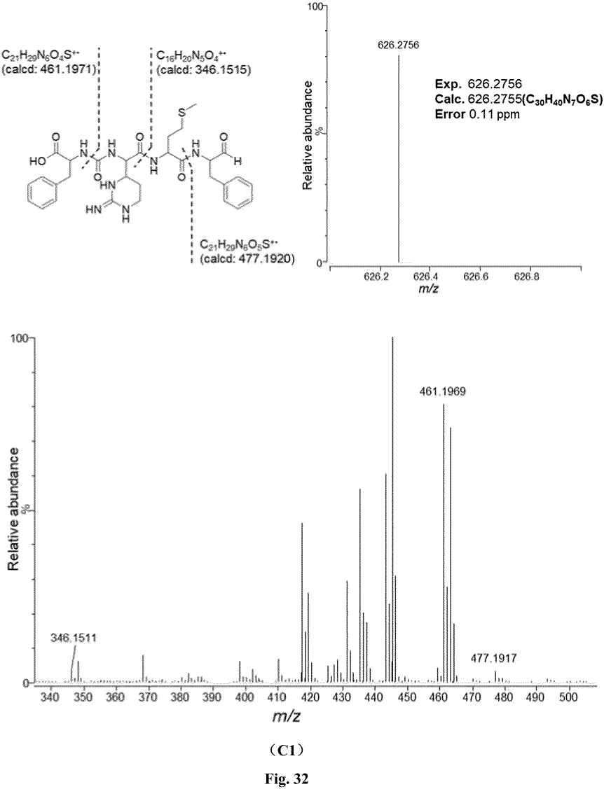

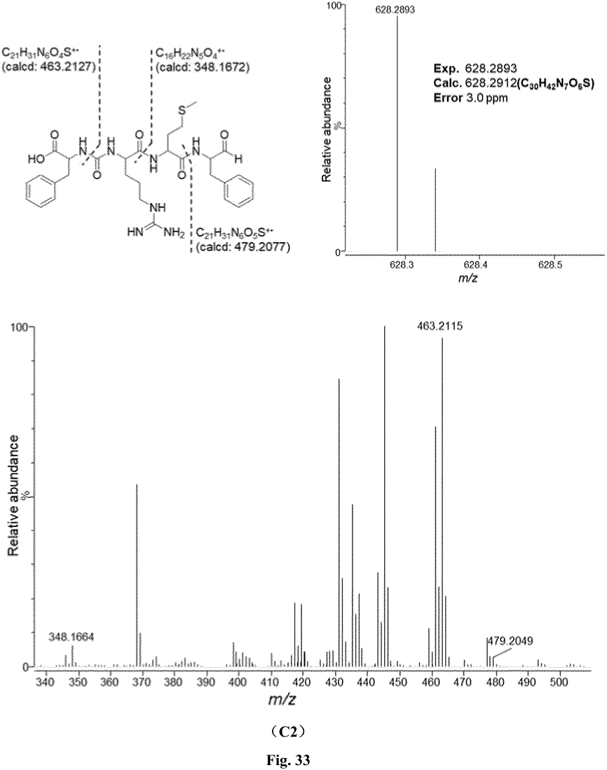

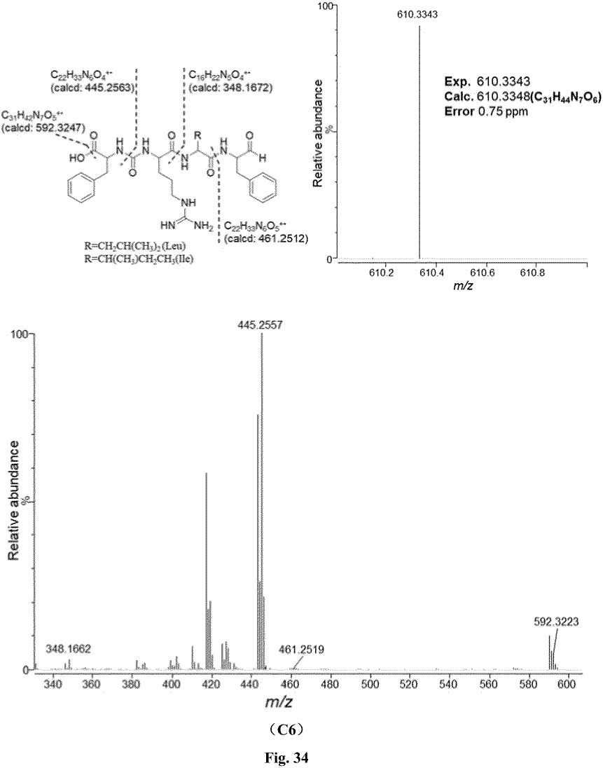

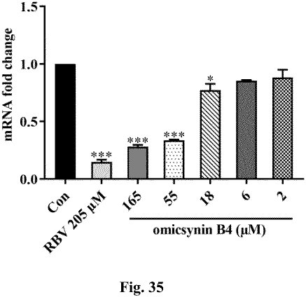

The HRMS analysis data of compounds A6, B1, B2, B3, B5, B6, C1, C2, C6 are shown in FIGS. 26~34.

TABLE 8

NMR data of compounds Omicsynin A1 and Omicsynin A2 (600 MHz, DMSO-d6)

| Omicsynin A1 | | | Omicsynin A2 | | |
|---|---|---|---|---|---|
| No. | δ_{H} | δ_{C},type | No. | δ_{H} | δ_{C},type |
| 1 | | 173.5, C | 1 | | 173.8, C |
| 2 | 4.28, m | 54.2, CH | 2 | 4.30, m | 54.1, CH |
| 3 | 3.01, dd (13.8, 6.0) | 37.5, CH₂ | 3 | 2.98, dd | 37.5, CH₂ |
| | 2.86, dd (13.8, 5.4) | | | 2.86, dd | |
| 4 | | 137.4, C | 4 | | 137.4, C |
| 5 | 7.16, ov^{b} | 129.2, CH | 5 | 7.16, ov^{b}. | 129.1, CH |
| 6 | 7.23, ov | 128.1, CH | 6 | 7.22, ov | 128.0, CH |
| 7 | 7.16, ov | 126.4, CH | 7 | 7.16, ov | 126.3, CH |
| 8 | 7.23, ov | 128.1, CH | 8 | 7.22, ov | 128.0, CH |
| 9 | 7.16, ov | 129.2, CH | 9 | 7.16, ov | 129.1, CH |
| NH | 6.46, d (7.8) | | NH | 6.32 | |
| 10 | | 157.3, C | 10 | | 157.2, C |
| 11 | 4.36, br t (7.8) | 54.5, CH | 11 | 4.11, m | 52.4, CH |
| 12 | 3.46, m | 50.9, CH | 12 | 1.57, m | 29.4, CH₂ |
| | | | | 1.43, m | |
| 13 | 1.60, m,1.67, m | 20.7, CH₂ | 13 | 1.43, m | 24.7, CH₂ |
| 14 | 3.28, m | 36.0, CH₂ | 14 | 3.07, m | 40.4, CH₂ |
| | 3.15, m | | | | |
| NH-12 | 7.53, br s | | | | |
| 15 | | 153.8, C | 15 | | 156.7, C |
| 16 | | 169.5, C | 16 | | 172.0, C |
| NH-14 | 8.12, br s | | NH-14 | 7.57 | |
| NH-11 | 6.70, d (8.4) | | | | |
| 17 | 4.28, m | 52.6, CH | 17 | 4.25, m | 52.0, CH |
| 18 | 1.86, m, 1.77, m | 31.8, CH | 18 | 1.82, m, 1.74, m | 32.0, CH₂ |
| 19 | 2.40, m | 29.4, CH₃ | 19 | 2.36, m | 30.1, CH₃ |
| 20 | 2.01, s | 14.6 CH₃ | 20 | 1.99, s | 14.6, CH₃ |
| 21 | | 171.0, C | 21 | | 170.5, C |
| NH-17 | 8.31, d (7.2) | | NH-17 | 7.98, d (7.2) | |
| 22 | 3.90, m | 52.6, CH | 22 | 3.87, m | 52.3, CH |
| 23 | 2.80, dd (13.8, 6) | 36.3, CH₂ | 23 | 2.80, dd (13.2, 5.4) | 36.4, CH₂ |
| | 2.64, dd (13.8, 7.2) | | | 2.63, dd (13.2, 8.4) | |
| 24 | | 138.8, C | 24 | | 138.9, C |
| 25 | 7.19, ov | 129.1, CH | 25 | 7.19, ov | 129.1, CH |
| 26 | 7.25, ov | 128.0, CH | 26 | 7.25, ov | 128.0, CH |
| 27 | 7.19, ov | 125.9, CH | 27 | 7.19, ov | 125.9, CH |
| 28 | 7.25, ov | 128.0, CH | 28 | 7.25, ov | 128.0, CH |
| 29 | 7.19, ov | 129.1, CH | 29 | 7.19, ov | 129.1, CH |
| 30 | 3.31, d (5.4) | 62.1, CH₂ | 30 | 3.29, ov | 62.2, CH₂ |
| NH-22 | 7.96, d (8.4) | | NH-22 | 7.74, d (8.4) | |

### Example 8. Determination of Antiviral Activity of the Omicsynins

### Determination of the Inhibitory Activity of the Omicsynins Against Influenza Virus H3N2

The inhibitory effect of some omixins on influenza virus (H3N2) strains was determined by CPE method, and the activity of ribavirin (RBV) was also determined.(shown in Table 9)

TABLE 9

the inhibitory activity of the Omicsynins against influenza virus H3N2
A/Wuhan/359/1995 (H3N2)

| Sample information | TC₅₀ (µM) | IC₅₀ (µM) | SI |
|---|---|---|---|
| Omicsynin A1 | > 318.83 | 318.83 ± 0 | > 1.00 |
| Omicsynin A2 (2) | > 794.53 | 207.77 ± 56.00 | > 3.82 |
| Omicsynin A3 (3) | > 335.97 | 87.85 ± 23.68 | > 3.82 |
| Omicsynin A4 (4) | > 83.71 | > 83.71 | - |
| Omicsynin B1 (7) | > 315.31 | 0.89 ± 0.20 | - > 352.94 |
| Omicsynin B2 (8) | > 314.32 | 1.00 ± 0.22 | > 312.78 |
| Omicsynin B3 (9) | > 332.06 | 3.34 ± 0.13 | > 99.34 |
| Omicsynin B4 (10) | > 330.96 | 2.43 ± 0.71 | > 136.05 |
| antipain | > 330.96 | 3.16 ± 0.94 | > 104.90 |
| chymostatin | > 329.33 | 329.33 ± 0 | > 1.00 |
| RBV | > 819.00 | 13.16 ± 3.11 | > 62.24 |

Sample description:
RBV, ribavirin injection was purchased from Tianjin Jinyao Group Hubei Tianyao Pharmaceutical Co., Ltd., the batch number is 31712252, and the specification is 100 mg/ml,
Antipain (#37682-72-7, 5 mg) was purchased from Shanghai Yifei Biotechnology Co., Ltd., which is equivalent to the monomeric compound Omicsynin B4 obtained from the fermentation sample of Streptomyces CPCC 200451 described in the present disclosure,
Chymostatin (#9076-44-2, 5 mg) was purchased from Sigma-Aldrich Company, the reagent contains three compounds Chymostatin A, B, C, which are equivalent to the compounds Omicsynin C3 and C5 described in the present disclosure,

### Activity Assays Were Repeated Three Times and Results Were Expressed as Mean ± SD.

### Determination of the Inhibitory Activity of the Omicsynins Against Coronavirus HCoV-229E

1. The steps of the anti-HCoV-229E virus activity assay of cytopathic effect (CPE) inhibition assay:
(1) the passaged hepatocyte Huh7.5 cells seeded in 96-well plates at 37° C. overnight (1×10⁴ cells/well) ;
(2) Infect cells with the virus at 100 times 50% tissue culture infective dose (TCID50) for 2 h (for HCoV- 229E, the test compound was added simultaneously or administration 2 h after infection), The test compounds are diluted three times for 8 doses, and the positive control drug Ribavirin Injection was diluted to the required concentration when used,
(3) Two parallel wells were set for each dose, and the results were observed when the lesions in the control group reached the CPE evaluation standard 4+. The 50% inhibitory concentration (IC50) was determined by the Reed and Muench method, The 50% toxicity concentration (TC50) of the test samples and positive compounds was also evaluated by the CPE assay. The selectivity index (SI) was calculated as the ratio of TC50/IC50 (shown in Table 10).

$${\text{IC}}_{50} = \underline{\text{AntiLog}} \left(\text{A} + \frac{\text{B} - 50}{50 - \text{C}} \times \text{D}\right)$$

Where: A=drug concentration with cumulative inhibition rate<50%,
B=inhibition rate with cumulative inhibition rate>50%,
C=inhibition rate with cumulative inhibition rate<50%,
D=log dilution factor
CPE evaluation criteria: the proportion of cell death was marked as 4+ (75% to 100% of cell death), 3+ (50% to 75% of cell death), 2+ (25% to 50% of cell death), 1+ (cell death ratio 0-25%), 0+ (all cells survive).

The experiments were repeated more than 2 times, and representative results are given.

TABLE 10

the inhibitory activity of the Omicsynins against coronavirus HCoV-229E
HCoV-229E

| Sample information | TC₅₀ (µM) | IC₅₀ (µM) | SI |
|---|---|---|---|
| Omicsynin A1 (1) | > 159.41 | 45.04 ± 7.03 | > 3.54 |
| Omicsynin A2 (2) | > 397.27 | 171.85 ± 2.10 | > 0.92 |
| Omicsynin A3 (3) | > 167.98 | 57.41 ± 15.62 | > 2.93 |
| Omicsynin A4 (4) | > 41.86 | > 41.86 | - |
| Omicsynin B1 (7) | > 157.65 | 1.35 ± 0.28 | > 117.19 |
| Omicsynin B2 (8) | > 157.16 | 1.53 ± 0.32 | > 102.90 |
| Omicsynin B3 (9) | > 166.03 | 1.19 ± 0.51 | > 139.53 |
| Omicsynin B4 (10) | > 165.48 | 0.89 ± 0.22 | > 186.34 |
| antipain | > 165.48 | 1.57 ± 0.46 | > 105.63 |
| chymostatin | > 164.66 | 23.74 ± 5.66 | > 6.93 |
| RBV | > 409.50 | 25.14 ± 6.10 | > 16.29 |

Sample description: same as above.

### 3. Determination of the Inhibitory Activity of the Omicsynins Against Coronavirus HCoV-OC43

### qRT-PCR Analysis to Detect the mRNA Expression Level of HCoV-OC43 N Protein in C3A Cells:

The passaged human hepatoblastoma cell line C3A were seeded in 12-well plates (3.5×105 cells/well), incubated and treated with the indicated concentrations of compounds for 24 hours. Total RNA from infected cells was extracted using the RNeasy Mini kit (QIAGEN). The mRNA expression level of HCoV-OC43 N protein was measured by the ABI 7500 Rapid RT-PCR System (Applied Biosystems) using the TransScriptTM Taqman One-Step qRT-PCR SuperMix Kit (TransGen Biotech), and was corrected for the GAPDH expression level. Using the software GraphPad Prism 8 to calculate the IC50 of the compounds against coronavirus HCoV-OC43.

### Determination of the Activity of Omicsynin B4 Against Coronavirus HCoV-OC43

The results showed that the IC50 of Omicsynin B4 for inhibitory activity of coronavirus HCoV-OC43 was 28.67 µM, and the IC50 of ribavirin (RBV) was determined at the same time, as shown in FIG. 35.

The above results showed that omicxin compounds also have a good inhibitory effect on coronavirus.

Finally, it should be noted that the above embodiments are only used to understand the essence of the present disclosure, and are not used to limit the protection scope of the present disclosure.

What is claimed is:

1. Uses of a group of peptide derivative Omicsynins for the treatment of a virus infection, wherein the chemical formula of the peptides derivative Omicsynins is shown in formula (1), 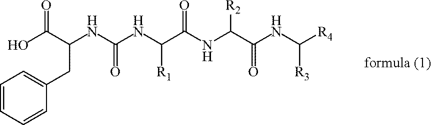
wherein R
₁-R₄ are shown in the following table,

| | | | |
|---|---|---|---|
| NO. | substituent 1 | substituent 2 | substituent 3 |
| R₁ | 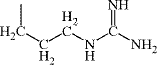 | 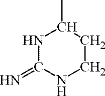 | Basic amino-acid side chains including lysine, histidine, citrulline residues, |
| | | | 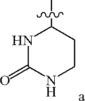 |
| | | | and etc. |
| R₂ | 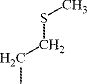 | CH(CH₃)₂ CH(CH₃)CH₂CH₃ [symbol] -CH₂CH(CH₃)₂ | Neutral amino-acid side chain including tryptophan, serine, threonine, cysteine residues and etc. |
| R₃ | 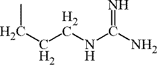 | 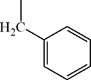 | R₃ including tyrosine, lysine, histidine, |
| | | | 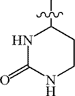 |
| | | | citrulline residues, 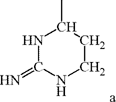 and etc. |
| R₄ | CH₂OH | CHO | R₄ including —CH₂NH₂¸CH═NH¸CH═NOH¸COOH¸ COORs (R₅ represents alkyl groups containing 1-3 carbons) ¸CONH₂ and |
| | | | etc. |

wherein the virus is influenza virus or coronavirus.

2. (canceled)

3. (canceled)

4. A group of peptide derivative Omicsynins, Omicsynin, B1, B2, B3, B5, B6, the general structural formula is shown in formula (1), 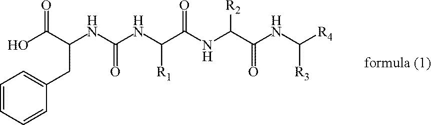
wherein the substituents of R
₁~R₄ of each compound are shown in the following table:

| | | |
|---|---|---|
| Name | R₁ | R₂ |
| Omicsynin B1 | 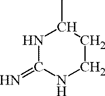 | 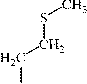 |
| Omicsynin B2 | 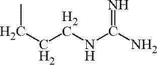 | 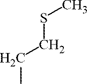 |
| Omicsynin B3 | 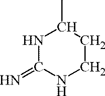 | CH (CH₃) ₂ |
| Omicsynin B5 | 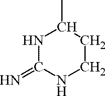 | CH(CH₃)CH₂CH₃ Or —CH₂CH(CH₃)₂ |
| Omicsynin B6 | 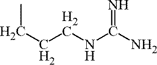 | CH(CH₃)CH₂CH₃ Or —CH₂CH(CH₃)₂ |
| Omicsynin B1 | 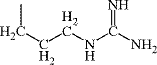 | CHO |
| Omicsynin B2 | 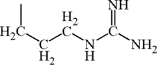 | CHO |
| Omicsynin B3 | 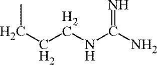 | CHO |
| Omicsynin B5 | 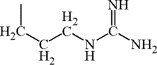 | CHO |
| Omicsynin B6 | 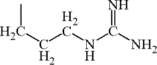 | CHO |

.

5. A biosynthetic gene clusters for the production of peptide derivative Omicsynins in a microorganism,
wherein the gene cluster is of Streptomyces sp. CPCC 200451 genome Chromosome 1: 7,822,964-7,875,615, with a full length of 52.6 kb, and wherein
the chemical formula of the peptides derivative Omicsynins is shown in formula (1): 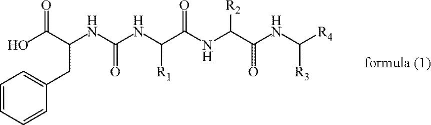
wherein R₁-R₄ are shown in the following table:

| | | | |
|---|---|---|---|
| NO. | substituent 1 | substituent 2 | substituent 3 |
| R₁ | 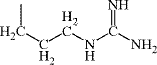 | 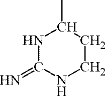 | Basic amino-acid side chains including lysine, histidine, citrulline residues, |
| | | | 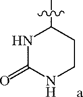 |
| | | | and etc. |
| R₂ | 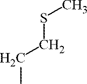 | CH(CH₃)₂¸ CH(CH₃)CH₂CH₃ [symbol] —CH₂CH(CH₃)₂ | Neutral amino-acid side chain including tryptophan, serine, threonine, cysteine residues and etc. |
| R₃ | 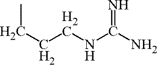 | 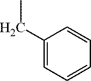 | R₃ including tyrosine, lysine, histidine, |
| | | | 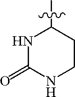 |
| | | | citrulline residues¸ |
| | | | 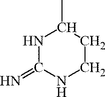 |
| | | | and etc. |
| R₄ | CH₂OH | CHO | R₄ including —CH₂NH₂¸CH═NH¸CH═NOH, —COOH¸ COORs (R₅ represents alkyl groups containing 1-3 carbons) ¸CONH₂ and etc. |

6. The biosynthetic gene cluster according to claim 5, wherein
gene 7094 and 7098 in said gene cluster are key biosynthetic genes of peptide derivative Omicsynins, and the amino acid sequences of the encoded proteins are shown in SEQ ID NO:1 and 2, and
gene 7102 in said gene cluster is a positive regulator of the peptide derivative Omicsynins synthetic gene, the amino acid sequence of the encoded protein is shown in SEQ ID NO:3, and its expression level is in proportion to the content of Omicsynins.

7. (canceled)

8. Use of the gene cluster of claim 9 to ptrpare peptides derivative Omicsynins showed in formula (1) by microbial fermentation: 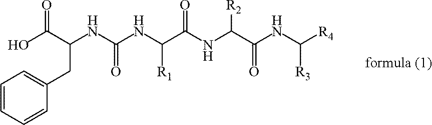
wherein R₁-R₄ are shown in the following table:

| | | | |
|---|---|---|---|
| NO. | substituent 1 | substituent 2 | substituent 3 |
| R₁ | 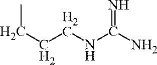 | 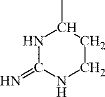 | Basic amino-acid side chains including lysine, histidine, citrulline residues, |
| | | | 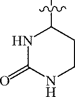 |
| | | | and etc. |
| R₂ | 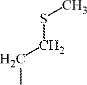 | —CH(CH₃)₂, —CH(CH₃)CH₂CH₃ [symbol] —CH₂CH(CH₃)₂ | Neutral amino-acid side chain including tryptophan, serine, threonine, cysteine residues and etc. |
| R₃ | 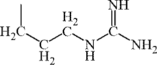 | 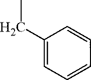 | R₃ including tyrosine, lysine, histidine, |
| | | | 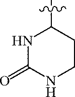 |
| | | | citrulline residues |
| | | | 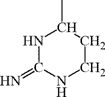 |
| | | | and etc. |
| R₄ | CH₂OH | CHO | R₄ including —CH₂NH₂¸CH═NH¸CH═NOH¸ —COOH¸ —COOR₅ (R₅ represents alkyl groups containing 1-3 carbons) ¸CONH₂ and etc. |

9. The biosynthetic gene cluster according to claim 5, wherein the gene cluster is between gene 7092-7102 (chromosome 1: 7,841,516-7,857,514), with a full length of 15.99 kb.


SEQUENCE LISTING (3 sequences)

SEQ ID NO: 1 - protein, 1035 aa, Streptomyces sp.
MFALSPSQEIVWLHEQMLPGSRAYNFTAVLDLWGTLDRDALHTGLAAVLDHHPGLRLELVPRAEGLPGQRVAEGCRPELRHSDFAAEADPEAAFRRLLAAEAEEPVDTERAPVLRWHLVRIAEDHHRLIHVEHHLVHDGHSFAILLRDLFTVYRAQVLGEPYELPAAPSYEEHARAAADLPRDRGLDFWTKELTGAPREVTLPGLARPGAERRHRGGQLRQAIGADLAERLRERSTADGHTPFSTLLALLAELLRRHSGHREMVIGTAVGNRPEGFEGTVGMFVNTVPLRLRLDPDATGTDAVDEATDVLMRALPYQDLPVQELTRRLGLHTSGADNPLWNVMFSAHDAALPDVQAPGLEVSLYEGFNTGTTRFDLDLVLLPDDRRVVGEKHGAAGMTLVWDYDADLFRESDAVLLSERLLELLRCYLDHPQAPIAELAPAPEADAEPTAVAAGATLDALDPAAAHDPTALALLAGTDRISYGELDRRVTELVERLRAAGVAPGQPVATVLPRGADSVVALLACLRHGAVYAPLSTEDPAARLGLLLERLSPALVLATRQSALALPAEGVTPALLDGPAFPKAEPAAVLDGAAYLIHTSGSTGVPKPVQVGREALAGYVAAITARYELTSADRVLLFARPSFDVALEEVLPALAVGAALVVPQREVPTGPELVAVLAARGVTVANLPTSYLLAVREDLREALRDGRWTPRLLVLGGERLPARALADLTDATTVLNAYGVTEATVTSTVHRVTAADVAEDGEIPLGTELDGVRMHVLDDTLRPLPAGCVGELVVAGPMLADGYVGLPEPTAARFVTVPALGGARVYRTGDRGYRDLDGRLWFLGRADNQVKLRGHRIELEEIEAAASAELGGRPCAVVLHTDPQAGPRLLGFLEGGGRVDQAAMHEALARRLPAGLVPASWTAVPKMPTLPGGKPDRAALARLVDESAAEPAEEQGPPSAFADPGQELIAAAWREVLGHDRFSADSHFFEVGGHSLLAAQLAAYLEPHLGSRPPLRTLFRHAVLADQARALTGDVK

SEQ ID NO: 2 - protein, 604 aa, Streptomyces sp.
MTTATLPALIARQAERQPDALAVTDGSTVLTYRELLDAAGRLAAVLTARGIGPGSAVGLLCARSTRLVVAQLAIWRAGGHVLPLDPAYPRPRIAEMTADAGAELVLGDKAMLAEAGLAEERTLVLTEHGTDAPDTSDAPGAAGPQAPDAAEPAAPALVFYTSGSTGRAKGVLVPHRAVADLVTAPDAIAVGPRDRVLFHSAVTFDASTFELWAPLARGAAVAVSPADRPTAEELGRDVERFGVTTLLLTSALFHHLAARQSRIFGVLRTLAVGGEVLSPEHAGAVLRAFPWLELVNVYGPTEATTFTTLHRVTPADCAGAVPIGRPFGGARALVLDERLAPVPTGTVGELWIAGDRLAHGYLNRPELTAERFRELPEVGRAYRSGDLVRQRPDGTLEFHGRTDEQVKLRGFRIEPGEIEHALLQLPEVAEAAVVVRRAGQHDAALTACLVLAEGAGQAVEPLRARLAERLPAHLVPTVWTVLDALPLTGSGKVDRRALAADDAVGKRPSGGGELTPIQQVVAEAWSRALERPITDPAADFLAEGGHSLLAMWVVDDLREDLGVELSLADFLDHPTIAGQAELLERALLAADGASGAEATRGAAR

SEQ ID NO: 3 - protein, 191 aa, Streptomyces sp.
MTDGSSERPLRADARRNRDKILAAAVRVYAEDGLDARMEHIAKAAGVGSATLYRNFPTREALIEAVYRQEVAQLCDAVPGLLAAQPPFQALFAWTRLFLDYVAAKSGMADALRAIAATGGNPHGRSRDMIGQALTELMDACAAAGAIRTDISPADMLAALEGIAVTSARPDQRERAERLLDLTLDGLTARS